(19) United States
(12) Patent Application Publication
(10) Pub. No.: US 2005/0089985 A1
(43) Pub. Date: Apr. 28, 2005

(54) Rescue of canine distemper virus from cdna

(75) Inventors: Christopher L. Parks, Boonton, NJ (US); Mohinderjit S. Sidhu, New City, NY (US); Pramila Walpita, Basking Ridge, NJ (US); Gerald R. Kovacs, Ridgewood, NJ (US); Stephen A. Udem, New York, NY (US)

Correspondence Address: WYETH;PATENT LAW GROUP, 5 GIRALDA FARMS, MADISON, NJ, 07940, US

(73) Assignee: American Cyanamid Company

(21) Appl. No.: 10/312,052

(22) PCT Filed: Jun. 22, 2001
(86) PCT No.: PCT/US01/20157

Related U.S. Application Data
(60) Provisional application No. 60/213,698, filed on Jun. 23, 2000.

(51) Int. Cl. C12N 7/00; C12N 15/86

(52) U.S. Cl. 435/235.1; 435/456

(57) ABSTRACT

This invention relates to a method for recombinantly producing via rescue canine distemper virus, a nonsegmented, negative-sense, single-stranded RNA virus, and immunogenic compositions formed therefrom. Additional embodiments relate to methods of producing the canine distemper virus as an attenuated and/or infectious viruses. The recombinant viruses can be prepared from cDNA clones, and, accordingly, viruses having defined changes, including nucleotide/polynucleotide deletions, insertions, substitutions and rearrangements, in the genome can be obtained.

### FIELD OF THE INVENTION

This invention relates to a method for recombinantly producing canine distemper virus, a nonsegmented, negative-sense, single-stranded RNA virus, and immunogenic compositions formed therefrom. Additional embodiments relate to methods of producing the canine distemper virus as an attenuated and/or infectious virus. The recombinant viruses are prepared from cDNA clones, and, accordingly, viruses having defined changes in the genome are obtained. This invention also relates to use of the recombinant virus formed therefrom as vectors for expressing foreign genetic information, e.g. foreign genes, for many applications, including imnnunogenic or pharmaceutical compositions for pathogens other than canine distemper, gene therapy, and cell targeting.

### BACKGROUND OF THE INVENTION

Enveloped, negative-sense, single stranded RNA viruses are uniquely organized and expressed. The genomic RNA of negative-sense, single stranded viruses serves two template functions in the context of a nucleocapsid: as a template for the synthesis of messenger RNAs (mRNAs) and as a template for the synthesis of the antigenome (+) strand. Negative-sense, single stranded RNA viruses encode and package their own RNA-dependent RNA Polymerase. Messenger RNAs are only synthesized once the virus has entered the cytoplasm of the infected cell, Viral replication occurs after synthesis of the mRNAs and requires the continuous synthesis of viral proteins. The newly synthesized antigenome (+) strand serves as the template for generating further copies of the (−) strand genomic RNA.

Canine distemper virus (CDV) is a member of the Morbillivirus genus (30). Like the other members of this group, including measles virus, Rinderpest virus, and Peste des petits ruminants virus among others, CDV is an enveloped RNA virus that contains a single-stranded, negative-sense genome of approximately 16 kilobases (4, 18, 25). The genome contains six non-overlapping gene regions, organized 3′-N-P-M-F-H-L-5′ that encode eight known viral polypeptides in infected cells. The viral polypeptides include: the nucleocapsid protein (N) that encapsidates viral genomic RNA; the matrix protein (M) that is a structural component of the virion; the fusion (F) and hemmagglutinin (H) envelope glycoproteins; the catalytic polymerase subunit (L); and three proteins encoded by the P gene. The P gene encodes the phosphoprotein (P) polymerase subunit and the nonstructural proteins (C and V) by making use of an alternative reading frame accessed from a downstream translation initiation codon (C) or a frameshift generated by mRNA editing (V).

CDV is best known for causing disease in dogs (4, 18). The virus is commonly spread by aerosol and initial infection occurs in the upper respiratory epithelium. The infection then spreads to the lymphoid tissues causing immunosuppression and further dissemination of the virus to many organs and cell types. Some animals recover from the disease, but within a few days to weeks, a relatively high number will develop an active infection of the central nervous system that leads to a progressive demyelinating disease that presents with neurological symptoms. This disease is studied as model for human demyenlating disorders (52, 57).

Although classically associated with infection of dogs, recent investigations with improved detection techniques have demonstrated that CDV infects a wide host range (4, 11, 18, 52). All canidae are susceptible including domestic and wild dogs, foxes, wolves and coyotes. CDV has also been linked to the deaths of large cats including lions and tigers in Africa and zoos in the United States. A CDV outbreak in seals has also been reported, and the virus is also known to cause disease in small carnivores like mink, ferrets and raccoons. CDV has even been considered a suspect in some human diseases like Paget's disease and multiple sclerosis (14, 19, 28). This relatively wide host range is rather unique among Morbilliviruses since the other members of this group display a restricted host range.

Live attenuated CDV vaccines have been effective in controlling the disease in domesticated dog populations but there is a need for additional vaccine research. The three prevalent vaccines cannot be used in all susceptible animal populations (4, 18, 52). Ferrets, foxes, some of the big cats, red pandas, and African wild dogs are susceptible to disease caused by vaccine strains, and this causes particular problems for zoos and wildlife parks trying to protect their animals from CDV infection. In addition the large host range of CDV suggests that there may be considerable potential for antigenic variation as well as adaptation to additional new hosts. Thus, vaccines that are safe for administration to a broader range of animals would be valuable, and it would be beneficial if these vaccines could be readily manipulated to take into account future antigenic variation.

New CDV vaccines are being investigated. For example, vaccines based on recombinant vaccinia virus or canarypox that express CDV glycoproteins have been tested in dogs and ferrets (34, 51) and these vaccines successfully elicit a protective immune response. However, it has yet to be determined if the duration of this immune response is equivalent to the response induced by conventional live CDV vaccines (4). DNA vaccines based on the CDV glycoproteins have been tested in mice. The immunized mice survived intracerebral challenge with a neurovirulent strain of CDV, but some mice may not have been completely protected from infection (48). In addition to testing these technologies, it may be desirable to attempt improvements in live attenuated CDV vaccines to enhance their safety in a broad range of hosts. The documented success of current live attenuated CDV vaccines in controlling distemper in domesticated dog populations (4, 18, 52) suggests that a modified and improved live attenuated CDV strain may still be one of the important options for vaccine development.

One important technology that could facilitate further development of a live attenuated CDV vaccine is the cDNA rescue technique that permits recovery of nonsegmented negative-strand RNA viruses from cloned DNAs (10, 31, 40, 42). Since it was first described (38, 44), this technology has been used with increasing frequency to derive attenuated strains, perform genetic analysis, and insert foreign genes in a variety of negative strand viruses (10, 31, 40, 42). Briefly, this technology enables the rescue of negative strand RNA viruses even though the genomic RNA of these viruses is not infectious. Rescue is accomplished by cloning the viral genomic cDNA into a plasmid vector that is designed to generate a precise copy of the viral genome in transfected cells expressing T7 RNA polymerase. This plasmid generally contains the cDNA flanked by a T7 RNA polymerase promoter at the 5′ end of the positive genomic strand and a ribozyme sequence at the 3′ end. Transcription initiation by T7 RNA polymerase forms the 5′ end of the viral genome while ribozyme cleavage of the primary T7 RNA polymerase-derived transcript forms the 3′ end. In addition to intracellular synthesis of the genome from a plasmid, T7 expression vectors containing the N, P and L genes are introduced into the cell to provide the minimal complement of traizs-acting factors necessary for initiation of virus rescue. A small percentage of cells cotransfected with the genomic cDNA clone and the expression plasmids for N, P and L will package a genomic transcript with N protein to form a nucleocapsid particle that then acts as a substrate for the viral polymerase composed of P and L proteins. After this step, the virus replication cycle can be initiated.

The polymerase complex actuates and achieves transcription and replication by engaging the cis-acting signals at the 3′ end of the genome, in particular, the promoter region. Viral genes are then transcribed from the genome template unidirectionally from its 3′ to its 5′ end. There is generally less mRNA made from the downstream genes (e.g., the polymerase gene (L)) relative to their upstream neighbors (i.e., the nucleoprotein gene (N)). Therefore, there is always a gradient of mRNA abundance according to the position of the genes relative to the 3′-end of the genome.

Molecular genetic analysis of such nonsegmented RNA viruses has proved difficult until recently because naked genomic RNA or RNA produced intracellularly from a transfected plasmid is not infectious. Currently, there are publications describing methods permit isolation of some recombinant nonsegmented, negative-strand RNA viruses (Schnell et al., 1994). These methods are referred to herein as “rescue”. The techniques for rescue of these different negative-strand viruses follows a common theme; however, each virus has distinguishing requisite components for successful rescue (41, 43, 44, 63, 64, 65, 66, 67, 68, 70 and 73). After transfection of a genomic cDNA plasmid, an exact copy of genome RNA is produced by the combined action of phage T7 RNA polymerase and a vector-encoded ribozyme sequence that cleaves the RNA to form the 3′ termini. This RNA is packaged and replicated by viral proteins initially supplied by co-transfected- expression plasmids. In the case of the canine distemper virus, a method of rescue has yet to be established and accordingly, there is a need to devise a method of canine distemper rescue. Devising a method of rescue for canine distemper virus is complicated by the absence of extensive studies on the biology of canine distemper virus, as compared with studies on other RNA viruses. Notably, CDV minireplicon studies have not been published and the minireplicon system essentially provides a starting point for developing virus rescue methods. Thus, no reagents have been available to establish a rescue system, such as N, P and L protein-expressing clones or a full-length genomic cDNA sequence. Additionally, cell culture conditions and transfection conditions required for effective minreplicon expression are unknown for CDV. A thorough understanding of these variables is important for successful rescue of any recombinant virus. Also, some strains of canine distemper virus do not grow efficiently in tissue culture systems. Despite the fact that a revised genomic sequence (at Genbank accession No. AF014953), which is incorporated herein by reference) has been available since 1998, no mimireplicon or virus rescue systems have been reported.

For successful cDNA rescue of canine distemper virus, numerous molecular events must occur after transfection, including: 1) accurate, full-length synthesis of genome or antigenome RNA by T7 RNA polymerase and 3′ end processing by the ribozyme sequence; 2) synthesis of viral N, P, and L proteins at levels appropriate to initiate replication; 3) the de novo packaging of genomic RNA into transcriptionally-active and replication-competent nucleocapsid structures; and 4) expression of viral genes from newly-formed nucleocapsids at levels sufficient for replication to progress.

The present invention provides for a rescue method of recombinantly producing canine distemper virus. The rescued canine distemper virus possesses numerous uses, such as antibody generation, diagnostic, prophylactic and therapeutic applications, cell targeting as well as the preparation of mutant virus and the preparation of immunogenic compositions and pharmaceutical compositions.

### SUMMARY OF THE INVENTION

The present invention provides for a method for producing a recombinant canine distemper virus comprising, in at least one host cell, conducting transfection of a rescue composition which comprises (i) a transcription vector comprising an isolated nucleic acid molecule which comprises a polynucleotide sequence encoding a genome or antigenome of a canine distemper virus and (ii) at least one expression vector which comprises at least one isolated nucleic acid molecule encoding the trans-acting proteins necessary for encapsidation, transcription and replication. The transfection is conducted under conditions sufficient to permit the co-expression of these vectors and the production of the recombinant virus. The recombinant virus is then harvested.

Additional embodiments relate to the nucleotide sequences, which upon mRNA transcription express one or more, or any combination of, the following proteins of the canine distemper virus: N, P, M F, H, L and the P,C, and V proteins (which are generated from the P gene of canine distemper virus as noted above). Related embodiments relate to nucleic acid molecules which comprise such nucleotide sequences. A preferred embodiment of this invention employs the nucleotide sequence of canine distemper virus as deposited with GenBank (accession number AF014953-SEQ ID NO. 1). Further embodiments relate to these nucleotides, the amino acids sequences of the above canine distemper virus proteins and variants thereof.

The protein and nucleotide sequences of this invention possess diagnostic, prophylactic and therapeutic utility for canine distemper virus. These sequences can be used to design screening systems for compounds that interfere or disrupt normal virus development, via encapsidation, replication, or amplification. The nucleotide sequence can also be used in immunogenic compositions for canine distemper virus and/or for other pathogens when used to express foreign genes.

In preferred embodiments, infectious recombinant virus is produced for use in immunogenic compositions and methods of treating or preventing infection by canine distemper virus and/or infection by other pathogens, wherein the method employs such compositions.

In alternative embodiments, this invention provides a method for generating recombinant canine distemper virus which is attenuated, infectious or both. The recombinant viruses are prepared from cDNA clones, and, accordingly, viruses having defmed changes in the genome can be obtained. Further embodiments employ the genome sequence employed herein to express foreign genes. Since we report here the complete cloning and sequencing of an entire cDNA clone of the Onderstepoort strain of canine distemper virus, the sequence is also an embodiment of the present invention.

This invention also relates to use of the recombinant virus formed therefrom as vectors for expressing foreign genetic information, e.g. foreign genes, for many applications, including immunogenic and pharmaceutical compositions for pathogens other than canine distemper virus, gene therapy, and cell targeting.

There are several compelling reasons why the successful rescue of canine distemper virus is very important for advancing technology and potential treatments. The ability to generate a recombinant CDV will facilitate the development of improved immunogenic compositions. The ability to generate a recombinant CDV will facilitate the development of CDV vectors. In addition, there are available animal models to study approaches for CDV-based immunogenic and pharmaceutical compositions and CDV-based viral vectors. The natural hosts, dogs and ferrets, could be used as experimental models for studying the genetic basis of CDV attenuation in the natural host organisms. Another benefit of a recombinant CDV is that since it is a neurotropic virus, the ability to generate a recombinant CDV will permit a genetic analysis of the neurotropism. Also, since CDV establishes acute and persistent infections, one can study the genetic analysis of persistent infection. Correspondingly, recombinant CDV can then be used to dissect the virus's ability to establish symptoms like those characteristic of human demyenlating diseases of the central nervous system.

Certain embodiments employ a laboratory-adapted strain of the Onderstepoort (17) of canine distemper virus. There are several advantages to using a laboratory-adapted strain as the initial model for rescue for canine distemper virus. First, the laboratory-adapted strain grows well in cultured cells. This characteristic will help promote successful rescue of recombinants. Second, the laboratory-adapted strain can grow well in a cell line qualified for vaccine production, such a Vero cells. Third, the laboratory-adapted strain is closely related to a vaccine virus (Onderstepoort) that has been used safely in dogs, thus, providing a likelihood that the recombinant virus will have also an attenuated phenotype. Fourth, if the laboratory-adapted recombinant virus requires further attenuation, the genome of the Onderstepoort strain can readily be characterized to identify attenuating mutations. Fifth, the laboratory-adapted strains possess an ability to grow in cultured cells, which aspect allows one to conduct the requisite initial studies in vitro rather than relying totally on animal model systems.

The above-identified embodiments and additional embodiments, which are discussed in detail herein, represent the objects of this invention.

### BRIEF DESCRIPTION OF THE FIGURES

FIG. 1 depicts a diagram showing the organization of the plasmid DNAs prepared for CDV rescue. FIG. 1A is a schematic diagram of the full-length CDV clone pBS-rCDV. The gene regions in the CDV genome are drawn as a black box with white letters and gene boundaries. The CDV leader and trailer sequences are drawn as open boxes at the termrini of the CDV genome. The genome is oriented in the plasmid vector to direct synthesis of a positive-sense RNA from the T7 RNA polymerase promoter (grey box) flanking the 5′ end of the genome. The hepatitis delta virus ribozyme sequence (hatched box in FIG. 1A; see Been et al., 1997 (5)) and two T7 RNA polymerase terminators (grey boxes) flank the 3′ end of the positive-sense cDNA. Restriction enzyme digestion sites used for cloning are indicated in italics.

FIG. 1B depicts the CDV minireplicon (pCDV-CAT). The minireplicon was prepared in the same vector used for the preparation of the viral cDNA clone. The CAT reporter gene flanked by the 107 nucleotide CDV leader and 106 nucleotide trailer (open boxes) was inserted between the NotI and NarI sites (Methods). The orientation of the minireplicon cDNA results in a negative-sense minireplicon RNA after T7 RNA polymerase transcription.

FIG. 1C depicts T7 RNA polymerase-dependent plasmid vectors (29) that were prepared to direct expression of the N, P or L genes in cells infected with MVA/T7 (61). The cDNA insert is cloned 3′ of an internal ribosome entry site (IRES) to facilitate translation of the T7 RNA polymerase transcript. A stretch of 50 adenosine residues is located at the 3′ end followed by a T7 RNA polymerase termninator.

FIG. 2A is an autoradiogram displaying the results of CAT assays performed to quantitate CDV-CAT minireplicon expression experiments as described in Example 3.1.1. In 2A, cells were transfected with 20 μg of minreplicon RNA and CDV-CAT minireplicon activity was driven by infection with CDV. The assay in Lane 1 was from a negative control that was not infected with CDV. Lane 2 illustrates the level of specific minireplicon activity driven by CDV infection.

FIG. 2B is an autoradiogram displaying the results of CAT assays were performed to quantitate CDV-CAT minireplicon expression experiments as described in Example 3.1.2. In 2B, cells were transfected with CDV minireplicon RNA (20 μg) plus T7 expression plasmids pCDV-N (1 μg), pCDV-P protein (1 μg) and pCDV-L (mass indicated in figure). Negative controls are shown in lane 1 (no N, P or L expression vectors) and lane 2 (no L expression vector). Lanes 3-5 were from identical transfections except that increasing amounts of L expression vector were used in these transfections.

FIG. 3A is a fluorescent image displaying the results of CAT assays for CDV-CAT minireplicon activity after transfection of pCDV-CAT plasmid DNA, as described in Example 3.1.3. The results in 3A demonstrate the effect of incubation temperature on minireplicon activity. Relative activity in FIG. 3A is expressed relative to the value given in lane 8.

FIG. 3B is an autoradiogram displaying the results of CAT assays for CDV-CAT minireplicon activity after transfection of pCDV-CAT plasmid DNA, as described in Example 3.1.4. FIG. 3B shows the beneficial effect of heat shock on minireplicon expression. CAT activity values in 3B are expressed relative to lane 2.

FIG. 4A depicts two representative plaques from the rescue of recombinant rCDV as described in Example 4.1. The first (left) plaque was rCDV rescued from the Onderstepoort strain genomic cDNA (pBS-rCDV). The second (right) plaque labeled rCDV-P/Luc/M is a recombinant strain that contains the luciferase gene described in FIG. 5A.

FIG. 4B depicts results from the analysis of RT/PCR-amplified products from rescued strains from the above experiments, as described in Example 4.2. Lanes 1-7 show the products of RT/PCR reactions amplified from the region between 1978 and 2604 on the CDV genome. A negative control in lane 1 (−L) was the RT/PCR result obtained using RNA derived from a coculture that originated from a rescue experiment that was performed without pCDV-L vector DNA. Lanes 3, 5, and 7 were negative controls in which the RT step of RT-PCR was omitted. Lanes 8-10 show the results of BstBI digestion on samples identical to the DNAs in lanes 2, 4 and 6. Digestion of the PCR fragment yields a doublet of approximately 315 base pairs and undigested fragment is 630 base pairs.

FIG. 5 contains six illustrations (A-F). Part (A) illustrates the structure of the CDV genome as it exists in the full-length cDNA clone. In part (B), part of the M/F intergenic region is shown (nucleotides 3320-3380) to illustrate how this region was altered to produce the multiple cloning sites found in the plasmid prCDV-mcs. Nucleotides shown in bold were changed to generate restriction sites. Parts (C-E) depict how the foreign genes were inserted into prCDV-mcs between the FseI and MluI sites. A synthetic copy of the M/F gene-end/gene-start signal was added to the 5′ end of the foreign gene during PCR amplification. In (F), the genomic location of the foreign gene (X) is illustrated on the CDV genome. Nomenclature: rCDV refers to recombinant viral strains; prCDV refers to plasmids (pBS-rCDV) containing the viral cDNA sequence.

FIG. 6 depicts the entire nucleotide sequence for a cDNA clone of CDV (SEQ ID NO 2).

FIG. 7 depicts the entire sequence for CDV full-length genomic clone (CDV genome plus vector; CDV sequence 2199-17888; total length 18826 base pairs), SEQ ID NO 3.

FIG. 8 is depicts the Western Blot Analysis of Proteins found in Extracts from Cells Infected with rCDV and rCDV-HBsAg Strains, pursuant to Example 5(c). Note that, rCDV-HBsAg-1, 2, and 3 were isolated from independent transfections performed with plasmid prCDV-HBsAg.

FIG. 9 depicts CPV VP2 coding region nucleotide sequence (SEQ ID NO 4)

FIG. 10 depicts the CPV VP2 predicted amino acid sequence (SEQ ID NO 5).

### DETAILED DESCRIPTION OF THE INVENTION

As noted above, the present invention relates to a method of producing recombinant canine distemper virus (CDV). Such methods in the art are referred to as “rescue” or reverse genetics methods. Several rescue methods for different nonsegmented, negative-strand viruses are disclosed (See 40, 41, 43, 44, 63, 64, 65, 66, 67 68, and 70). Additional publications on rescue include published International patent application WO 97/06270 for measles virus and other viruses of the subfamily Paramyxovirinae, and for RSV rescue, published International patent application WO 97/12032.

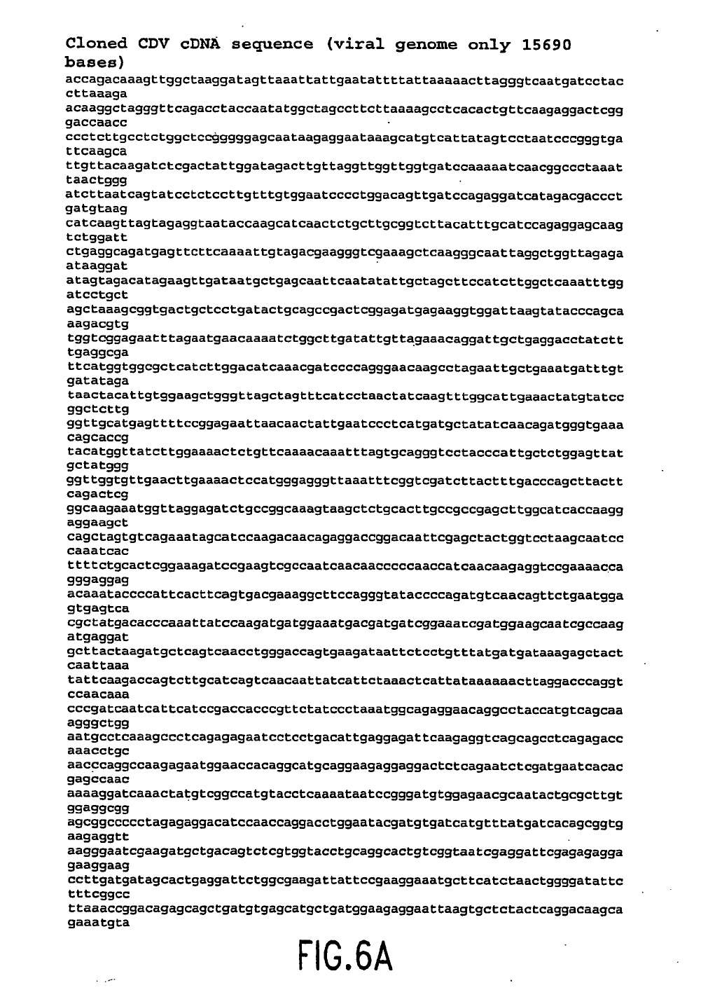

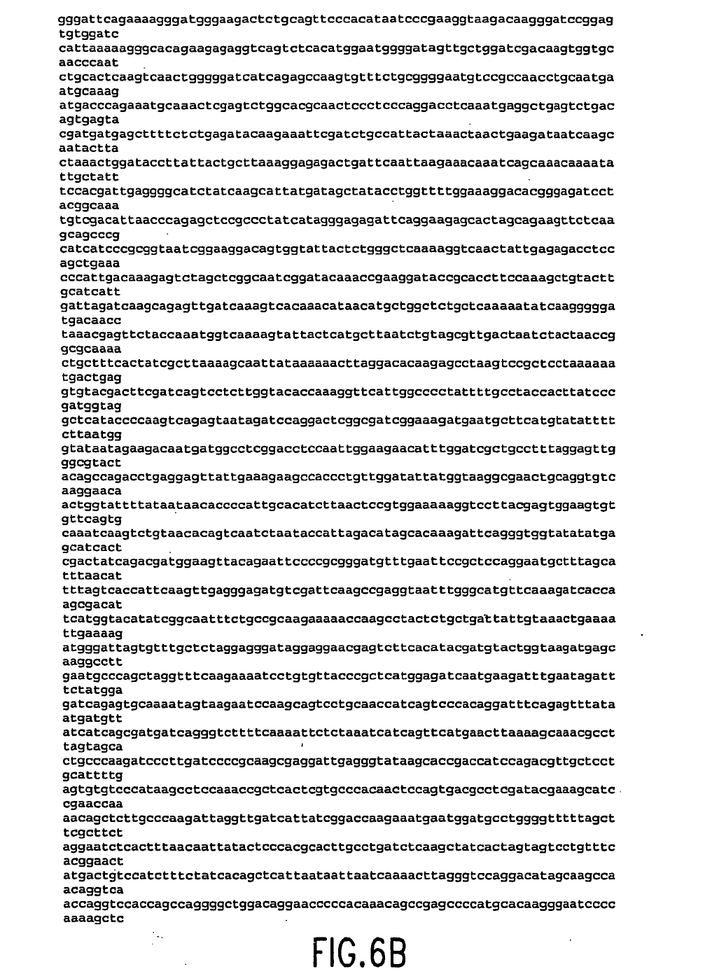

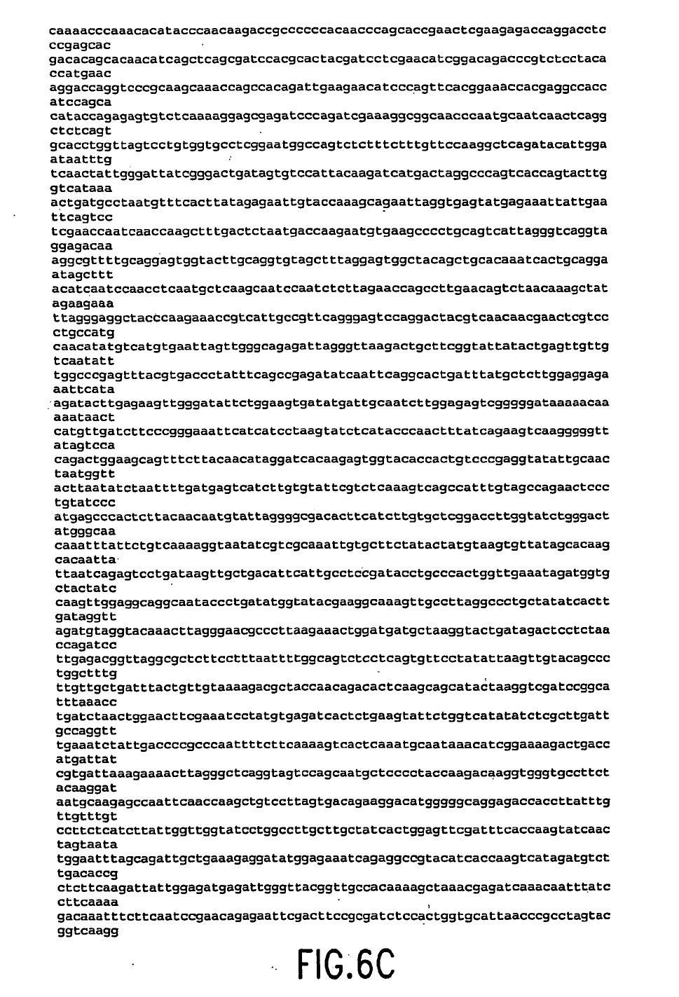

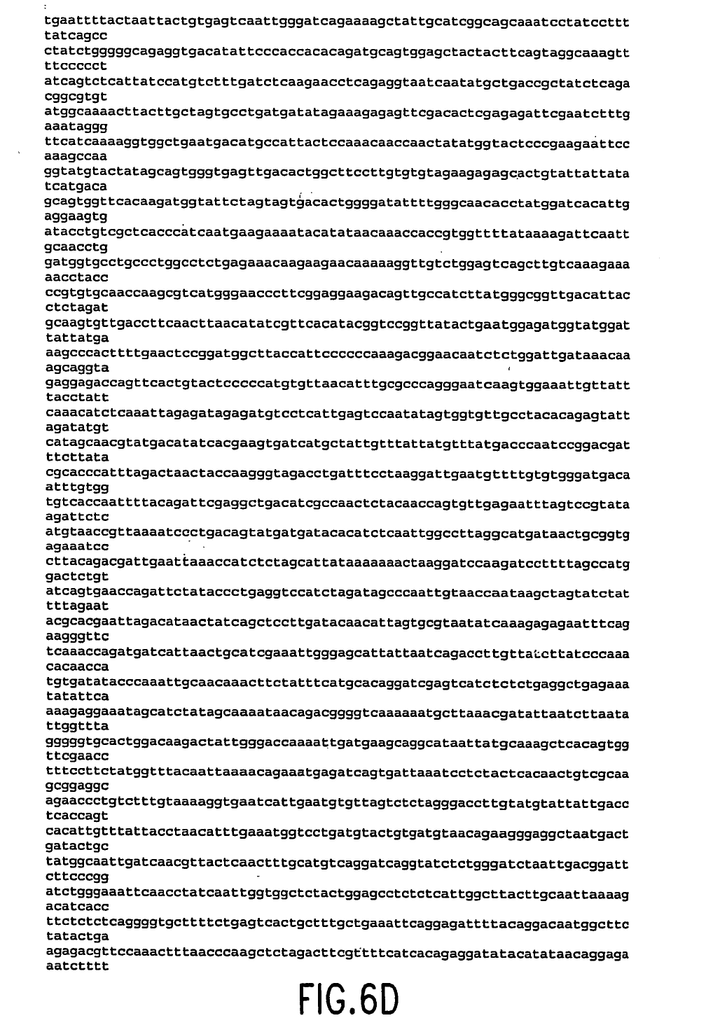

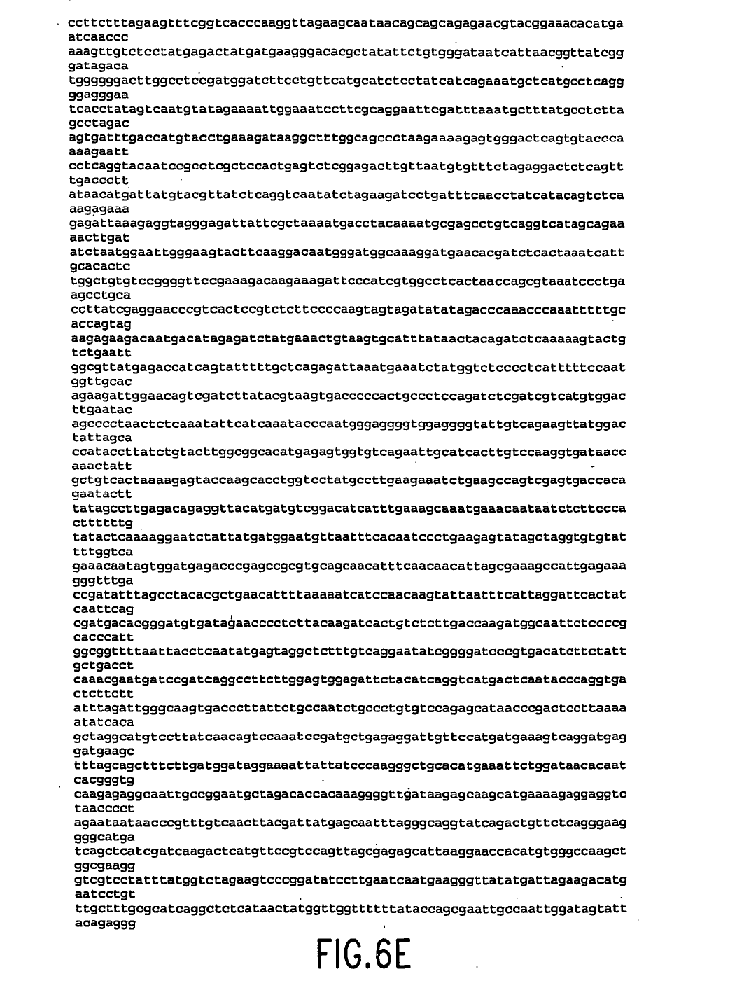

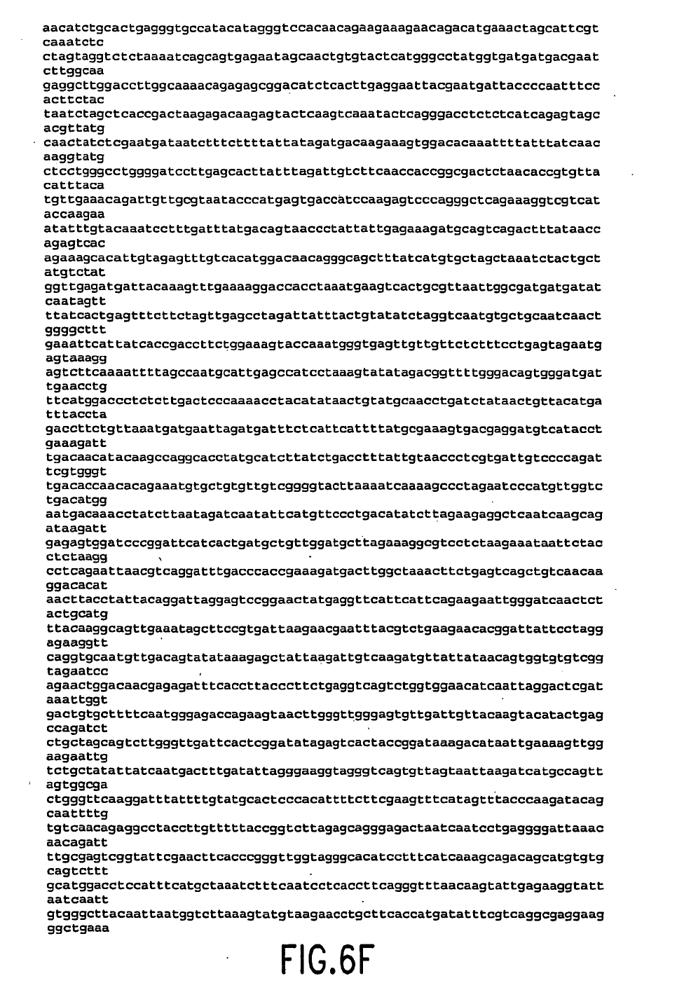

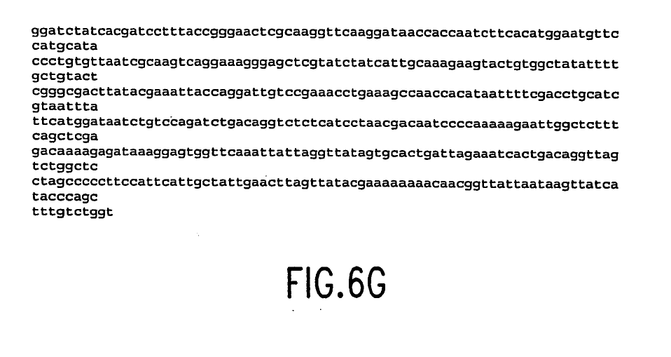

Further embodiments of this invention relate to rescue methods and compositions that employ a polynucleotide sequence encoding the genome or antigenome of canine distemper virus or proteins thereof, as well as variants of such sequences. These variant sequences, preferably, hybridize to polynucleotides encoding one or more canine distemper proteins, such as the polynucleotide sequence of Genbank Accession Number AF014953 or SEQ ID NO. 1 (of FIG. 6), under high stringency conditions. For the purposes of defining high stringency southern hybridization conditions, reference can conveniently be made to Sambrook et al. (1989) at pp. 387-389 which is herein incorporated by reference, where the washing step at paragraph 11 is considered high stringency. This invention also relates to conservative variants wherein the polynucleotide sequence differs from a reference sequence through a change to the third nucleotide of a nucleotide triplet. Preferably these conservative variants function as biological equivalents to the canine distempers virus reference polynucleotide sequence.

This invention also relates to nucleic acid molecules comprising one or more of such polynucleotides. As noted above, a given nucleotide recombinant sequence may contain one or more of the genomes of varying strains of Canine distemper virus. Specific embodiments employ the nucleotide sequence of SEQ ID. NO 1 or nucleotide sequences, which when transcribed, express one or more of the canine distemper virus proteins (N, P-P/C/V, M, F, H, and L).

Further embodiments employ the amino acid sequences for the canine distemper virus proteins (N, P-P/C/V, M, F, H, and L), for which the translated sequences are in Genbank AF014953, and also to fragments or variants thereof. Preferably, the fragments and variant amino acid sequences and variant nucleotide sequences expressing canine distemper virus proteins are biological equivalents, i.e. they retain substantially the same function of the proteins in order to obtain the desired recombinant canine distemper virus, whether attenuated, infectious or both. Such variant amino acid sequences are encoded by polynucleotide sequences of this invention. Such variant amino acid sequences may have about 70% to about 80%, and preferably about 90%, overall similarity to the amino acid sequences of the canine distemper virus protein. The variant nucleotide sequences may have either about 70% to about 80%, and preferably about 90%, overall similarity to the nucleotide sequences which, when transcribed, encode the amino acid sequences of the canine distemper virus protein or a variant amino acid sequence of the canine distemper virus proteins. Exemplary nucleotide sequences for canine distemper virus proteins N, P-P/C/V, M, F, H, and L are set forth for which the translated sequences are in Genbank AF014953, which sequences are incorporated herein.

The biological equivalents can be obtained by generating variants of the nucleotide sequence or the protein sequence. The variants can be an insertion, substitution, deletion or rearrangement of the template sequence. Variants of a canine distemper polynucleotide sequence can be generated by conventional methods, such as PCR mutagenesis, amino acid (alanine) screening, and site specific mutagenesis. The phenotype of the variant can be assessed by conducting a rescue with the variant to assess whether the desired recombinant canine distemper virus is obtained or the desired biological effect is obtained, if the ability to interrupt the ability to rescue a canine distemper virus is to be assessed. The variants can also be assessed for antigenicity if the desired use is an immunogenic composition.

Amino acid changes may be obtained by changing the codons of the nucleotide sequences. It is known that such changes can be obtained based on substituting certain amino acids for other amino acids in the amino acid sequence. For example, through substitution of alternative amino acids, small conformational changes may be conferred upon protein that may result in a reduced ability to bind or interact with other proteins of the canine distemper virus. Additional changes may alter the level of attenuation of the recombinant canine distemper virus.

One can use the hydropathic index of amino acids in conferring interactive biological function on a polypeptide, as discussed by Kyte and Doolittle (69), wherein it was found that certain amino acids may be substituted for other amino acids having similar hydropathic indices and still retain a similar biological activity. Alternatively, substitution of like amino acids may be made on the basis of hydrophilicity, particularly where the biological function desired in the polypeptide to be generated is intended for use in immunological embodiments. See, for example, U.S. Pat. No. 4,554,101 (which is hereby incorporated herein by reference), which states that the greatest local average hydrophilicity of a “protein,” as governed by the hydrophilicity of its adjacent amino acids, correlates with its immunogenicity. Accordingly, it is noted that substitutions can be made based on the hydrophilicity assigned to each amino acid.

In using either the hydrophilicity index or hydropathic index, which assigns values to each amino acid, it is preferred to introduce substitutions of amino acids where these values are ±2, with ±1 being particularly preferred, and those within ±0.5 being the most preferred substitutions.

Preferable characteristics of the canine distemper virus proteins, encoded by the nucleotide sequences of this invention, include one or more of the following: (a) being a membrane protein or being a protein directly associated with a membrane; (b) capable of being separated as a protein using an SDS acrylamide (10%) gel; and (c) retaining its biological function in contributing to the rescue production of the desired recombinant canine distemper virus in the presence of other appropriate canine distemper virus proteins.

With the above nucleotide and amino acid sequences in hand, one can then proceed in rescuing canine distemper virus. Canine distemper rescue is achieved by conducting transfection, or transformation, of at least one host cell, in media, using a rescue composition. The rescue composition comprises (i) a transcription vector comprising an isolated nucleic acid molecule which comprises at least one polynucleotide sequence encoding a genome or antigenome of canine distemper virus and (ii) at least one expression vector which comprises one or more isolated nucleic acid molecule(s) encoding the trans-acting proteins necessary for encapsidation, transcription and replication; under conditions sufficient to permit the co-expression of said vectors and the production of the recombinant virus. By antigenome is meant an isolated positive message sense polynucleotide sequence which serves as the template for synthesis of progeny genome. Preferably, a polynucleotide sequence is a cDNA which is constructed to provide upon transcription a positive sense version of the canine distemper genome corresponding to the replicative intermediate RNA, or antigenome, in order to minimize the possibility of hybridizing with positive sense transcripts of complementing sequences encoding proteins necessary to generate a transcribing, replicating nucleocapsid. The transcription vector comprises an operably linked transcriptional unit comprising an assembly of a genetic element or elements having a regulatory role in the canine distemper virus expression, for example, a promoter, a structural gene or coding sequence which is transcribed into canine distemper virus RNA, and appropriate transcription initiation and termination sequences.

The transcription vector is co-expressed with canine distemper virus proteins, N, P and L, which are necessary to produce nucleocapsid capable of RNA replication, and also render progeny nucleocapsids competent for both RNA replication and transcription. The N, P and L proteins are generated from one or more expression vectors (e.g. plasmids) encoding the required proteins, although one, or one or more, of these required proteins may be produced within the selected host cell engineered to contain and express these virus-specific genes and gene products as stable transformants. In a preferred embodiment, N, P and L proteins are expressed from an expression vector. More preferably, N, P and L proteins are each expressed from separate expression vectors, such as plasmids. In the latter instance, one can more easily control the relative amount of each protein that is provided during transfection, or transformation. Additional canine distemper virus proteins may be expressed from the plasmids that express for N, P or L, or the additional proteins can be expressed by using additional plasmids.

Although the amount of N, P and L will vary depending on the tolerance of the host cell for their expression, the plasmids expressing N, P and L are adjusted to achieve an effective molar ratio of N, P and L, within the cell. The effective molar ratio is a ratio of N, P and L that is sufficient to provide for successful rescue of the desired recombinant canine distemper virus. These ratios can be obtained based on the ratios of the expression plasmids as observed in minireplicon (CAT/reporter) assays. In one embodiment, the molecular ratio of transfecting plasmids pCDVN: pCDVP is at less than about 5:1 and pCDVP:pCDVL is less than about 15:1. Preferably, the molecular ratio of pCDVN: pCDVP is about 3:1 to about 1:3 and pCDVP:pCDVL is about 10:1 to about 1:5. More preferably, the ratio of pCDVN: pCDVP is about 2:1 and pCDVP:pCDVL is about 8:1 to about 1:1, with a most preferred ratio of pCDVN: pCDVP being about 1.2:1 and for pCDVP:pCDVL being about 5:1.

After transfection or transformation of a genomic cDNA plasmid along with canine distemper virus expression plasmids pCDVN, pCDVP and pCDVL, a precise copy of genome RNA is produced by the combined action of phage T7 RNA polymerase and a vector-encoded ribozyme sequence that cleaves the RNA to form the 3′ termini. This RNA is packaged and replicated by viral proteins initially supplied by co-transfected expression plasmids. In the case of the canine distemper virus rescue, a source that expresses T7 RNA polymerase is added to the host cell (or cell line), along with the source(s) for N, P and L. Canine distemper virus rescue is achieved by co-transfecting this cell line with a canine distemper virus genomic cDNA clone containing an appropriately positioned T7 RNA polymerase promoter and expression plasmids that encodes the canine distemper virus proteins N, P and L.

For rescue of canine distemper virus, a cloned DNA equivalent of the desired viral genome is placed between a suitable DNA-dependent RNA polymerase promoter (e.g., the T7 RNA polymerase promoter) and a self-cleaving ribozyme sequence (e.g., the hepatitis delta ribozyme) which is inserted into a suitable transcription vector (e.g a bacterial plasmid). This transcription vector provides the readily manipulable DNA template from which the RNA polymerase (e.g., T7 RNA polymerase) transcribes a single-stranded RNA copy of the viral antigenome (or genome) with the precise, or nearly precise, 5′ and 3′ termini. The orientation of the viral genomic DNA copy and the flanking promoter and ribozyme sequences determines whether antigenome or genome RNA equivalents are transcribed.

Accordingly, in the rescue method a rescue composition is employed. The rescue composition can be varied as desired for a particular need or application. An example of a rescue composition is a composition which comprises (i) a transcription vector comprising an isolated nucleic acid molecule which comprises a polynucleotide sequence encoding a genome or antigenome of canine distemper virus and (ii) at least one expression vector which comprises at least one isolated nucleic acid molecule encoding the trans-acting proteins necessary for encapsidation, transcription and replication. The transcription and expression vectors are selected such that transfection of the rescue composition in a host cell results in the co-expression of these vectors and the production of the recombinant canine distemper virus.

As noted above, the isolated nucleic acid molecule comprises a sequence that encodes at least one genome or antigenome of a canine distemper virus. The isolated nucleic acid molecule may comprise a polynucleotide sequence which encodes a genome, antigenome or a modified version thereof. In one embodiment, the polynucleotide encodes an operably linked promoter, the desired genome or antigenome, a self-cleaving ribozyme sequence and a transcriptional terminator.

In a preferred embodiment of this invention, the polynucleotide encodes a genome or anti-genome that has been modified from a wild-type canine distemper virus by a nucleotide insertion, rearrangement, deletion or substitution. It is submitted that the ability to obtain replicating virus from rescue may diminish as the polynucleotide encoding the native genome and antigenome is increasingly modified. The genome or antigenome sequence can be derived from that of any strain of canine distemper virus. The polynucleotide sequence may also encode a chimeric genome formed from recombinantly joining a genome or antigenome or genes from one or more heterologous sources.

Since the recombinant viruses formed by the methods of this invention can be employed as tools in diagnostic research studies or as therapeutic or prophylactic immunogenic and pharmaceutical compositions, the polynucleotide may also encode a wild type or any modified form of the canine distemper. In many embodiments, the polynucleotide encodes an attenuated, infectious form of the canine distemper virus. An attenuated form of the virus may result from mutations that occur within the coding regions of one or more genes as well as within one or more non-coding regions, i.e. intergenic regions of the genome. Several attenuating mutations are discussed in further detail, supra. For example, an attenuated form can be a polynucleotide that encodes a genome or antigenome of a canine distemper virus having at least one attenuating mutation in the 3′ genomic promoter region and having at least one attenuating mutation in the RNA polymerase gene, as described in Published International Patent Application WO 98/13501.

Modified forms of the polynucleotides may also encode a defective virus. The defective virus contains an alteration in the polynucleotide encoding CDV such that the recombinantly-produced virus is not replication competent. The mutation often occurs in, or at, one or more genes that encode a protein essential for replication of the virus. To obtain replication, the defective virus must be complemented with a host cell that contains the unmodified form (un-altered form) of the nucleotide sequence which may altered to render the virus defective. Such a host cell and cell line are termed a complementing cell or complementing cell line. The defective cells are preferably propagated in a complementing cell line in order to generate virus that is replication incompetent.

The present invention also relates to non-infectious alterations of a CDV polynucleotide sequence. For CDV, one may desire to alter a gene, nucleotide sequence that is involved in the production of infectious virus, but not involved in preventing replication of the viral genome. These alterations and CDV polynucleotides containing such are termed “non-infectious alterations and non-infectious CDV polynucleotides. The appropriate alteration, whether replication defective or non-infectious, may vary with the intended use, e.g. defective for replication in human cells versus canine or equine cells.

The altered sequence may be provided to the defective or non-infectious recombinantly-produced virus by complementing. Such complemented recombinant virus may also be used for pharmaceutical applications, such as gene delivery for gene therapy or as part of immunogenic compositions.

In addition to polynucleotide sequences encoding the modified forms of the desired canine distemper genome and antigenome as described above, the polynucleotide sequence may also encode the desired genome or antigenome along with one or more heterologous genes or a desired heterologous nucleotide sequence. Heterologous means that either the gene, or nucleotide sequence, which is inserted is not present in a recipient strain of CDV or the gene, or nucleotide sequence, is not present normally in the manner in which it is inserted into the CDV polynucleotide sequence. These variants are prepared by introducing selected heterologous nucleotide sequences into a polynucleotide sequence encoding a genome or antigenome of canine distemper. The desired heterologous sequence can be inserted within a non-essential or non-coding region of the canine distemper polynucleotide sequence, or inserted between a non-coding region and a coding region, or inserted at either end of the polynucleotide sequence. In alternative embodiments, a desired heterologous sequence is inserted within the non-coding region or in place of a coding region of a non-essential gene. The place of insertion can make use of the gradient expression characteristics of the canine distemper virus (25). Different levels of foreign antigen expression are readily examined in this type of rescue system by inserting the heterologous sequence in different genomic locations that take advantage of the natural 3′ to 5′ decreasing gradient of canine distemper virus.

The heterologous nucleotide sequence (e.g. gene) can vary as desired. Depending on the application of the desired recombinant virus, the heterologous nucleotide sequence may encode a co-factor, cytokine (such as an interleukin), a T-helper epitope, a restriction marker, adjuvant, or a protein of a different microbial pathogen (e.g. virus, bacterium, fungus or parasite), especially proteins capable of eliciting a protective inmmune response. It may be desirable to select a heterologous sequence that encodes an immunogenic portion of a co-factor, cytokine (such as an interleukin), a T-helper epitope, a restriction marker, adjuvant, or a protein of a different microbial pathogen (e.g. virus, bacterium or fungus) in order to maximize the likelihood of rescuing the desired canine distemper virus, or minireplicon virus vector. For example, in certain embodiments, the heterologous genes encode cytokines, such as interleukin-12, which are selected to improve the prophylatic or therapeutic characteristics of the recombinant virus or antigen expressed therefrom.

Antigens for se in the present invention may be selected from any antigen that is useful for a desired indication. The antigen may be added to a composition of this invention or expressed as a heterologous sequences from the recombinantly-produced canine distemper virus, as noted. One may select antigens useful against one or more pathogens, e.g. viruses, bacteria or fungi. A detailed list of potential pathogen targets as shown below.

Examples of such viruses include, but are not limited to, Human immunodeficiency virus, Simian immunodeficiency virus, Respiratory syncytial virus, Parainfluenza virus types 1-3, Herpes simplex virus, Human cytomegalovirus, Hepatitis A virus, Hepatitis B virus, Hepatitis C virus, Human papillomavirus, poliovirus, rotavirus, caliciviruses, Measles virus, Mumps virus, Rubella virus, adenovirus, rabies virus, rinderpest virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus and various Encephalitis viruses.

Examples of such bacteria include, but are not limited to, Haemophilus influenzae (both typable and nontypable), Haemophilus somnus, Moraxella catarrhalis, Streptococcus pneumnoniae, Streptococcus pyogenes, Streptococcus agalactiae, Streptococcus faecalis, Helicobacter pylori, Neisseria meningitidis, Neisseria gonorrhoeae, Chlamydia trachomatis, Chlamydia pneumoniae, Chlamydia psittaci, Bordetella pertussis, Salmonella typhi, Salmonella typhimurium, Salmonella choleraesuis, Escherichia coli, Shigella, Vibrio cholerae, Corynebacterium diphtheriae, Mycobacteriumn tuberculosis, Mycobacterium avium-Mycobacterium intracellulare complex, Proteus mirabilis, Proteus vulgaris, Staphylococcus aureus, Clostridium tetani, Leptospira interrogans, Borrelia burgdorferi, Pasteurella haemolytica, Pasteurella multocida, Actinobacillus pleuropneumoniae and Mycoplasma gallisepticum.

Examples of such fungi include, but are not limited to, Aspergillis, Blastonmyces, Candida, Coccidiodes, Cryptococcus and Histoplasnma.

Examples of such parasites include, but are not limited to, Leishmania major, Ascaris, Trichuris, Giardia, Schistosoma, Cryptosporidium, Trichomoinas, Toxoplasma gondii and Pneumocystis carinii.

Other types heterologous sequences may encode one or more peptides or polypeptides useful in eliminating or reducing diseased cells including, but are not limited to, those from cancer cells or tumor cells, allergens amyloid peptide, protein or other macromolecular components.

Examples of such cancer cells or tumor cells include, but are not limited to, prostate specific antigen, carcino-embryonic antigen, MUC-1, Her2, CA-125 and MAGE-3.

Examples of such allergens include, but are not limited to, those described in U.S. Pat. No. 5,830,877 and published International Patent Application Number WO 99/51259, which are hereby incorporated by reference, and include pollen, insect venoms, animal dander, fungal spores and drugs (such as penicillin). Such components interfere with the production of IgE antibodies, a known cause of allergic reactions.

Amyloid peptide protein (APP) has been implicated in diseases referred to variously as Alzheimer's disease, amyloidosis or amyloidogenic disease. The β-amyloid peptide (also referred to as Aβ peptide) is a 42 amino acid fragment of APP, which is generated by processing of APP by the β and γ secretase enzymes, and has the following sequence:

Asp Ala Glu Phe Arg His Asp Ser Gly Tyr Glu Val
His His Gln Lys Leu Val Phe Phe Ala Glu Asp Val
Gly Ser Asn Lys Gly Ala Ile Ile Gly Leu Met Val
Gly Gly Val Val Ile Ala.

In some patients, the amyloid deposit takes the form of an aggregated Aβ peptide. Surprisingly, it has now been found that administration of isolated Aβ peptide induces an immune response against the Aβ peptide component of an amyloid deposit in a vertebrate host (See Published International Patent Application WO 99/27944). Such Aβ peptides have also been linked to unrelated moieties. Thus, the heterologous nucleotide sequences of this invention include the expression of this Aβ peptide, as well as fragments of Aβ peptide and antibodies to Aβ peptide or fragments thereof. One such fragment of Aβ peptide is the 28 amino acid peptide having the following sequence (As disclosed in U.S. Pat. No. 4,666,829):

Asp Ala Glu Phe Arg His Asp Ser Gly Tyr Glu
Val His His Gln Lys Leu Val Phe Phe Ala Glu Asp
Val Gly Ser Asn Lys.

By expressing heterologous sequences, recombinant forms of canine distemper virus can be used in the same manner as an expression vector for the delivery of varied active ingredients, in the form of varied RNAs, amino acid sequences, polypeptides and proteins to an animal or human. The recombinant canine distemper virus can be used to express one or more heterologous genes (and even 3, 4, or 5 genes) under control of the virus transcriptional promoter. In alternative embodiments, the additional heterologous nucleic acid sequence may be a single sequence of up to 7 to 10 kb, which is expressed as a single extra transcriptional unit. Preferably, the Rule of Six (ref.6) is followed. In certain preferred embodiments this sequence may be up to 4 to 6 kb. One may also insert heterologous genetic information in the form of additional monocistronic transcriptional units, and polycistronic transcriptional units. Use of the additional monocistronic transcriptional units, and polycistronic transcriptional units should permit the insertion of more genetic information. In preferred embodiments, the heterologous nucleotide sequence is inserted within the canine distemper virus genome sequence as at least one polycistronic transcriptional unit, which may contain one or more ribosomal entry sites.

In alternatively preferred embodiments, the heterologous nucleotide sequence encodes a polyprotein and a sufficient number of proteases that cleaves said polyprotein to generate the individual polypeptides of the polyprotein.

The heterologous nucleotide sequence can be selected to make use of the normal route of infection of canine distemper virus, which enters the body through the respiratory tract and can infect a variety of tissues and cells, for example, salivary glands, lymphoid tissue, mammary glands, the testes and even brain cells. The heterologous gene may also be used to provide agents that can be used for gene therapy or for the targeting of specific cells. As an alternative to merely taling advantage of the normal cells exposed during the normal route of canine distemper infection, the heterologous gene, or fragment, may encode another protein or amino acid sequence from a different pathogen which, when employed as part of the recombinant canine distemper virus, directs the recombinant canine distemper virus to cells or tissue which are not in the normal route of canine distemper virus. In this manner, the recombinant canine distemper virus becomes a vector for the delivery of a wider variety of foreign genes, and accordingly, the delivery of numerous types of antigens. Our examples demonstrate that recombinant canine distemper virus can be used as an expression vector. The recombinant canine distemper virus expression vector may be used to deliver one or more antigens. Antigens from a variety of infectious agents (1, 7) may be selected for a desired application. One can selected an antigen that is useful against any of the following pathogens.

```
BOVINE
BRSV
BVD
Campylobacter
Haemophilus somnus
IBR
Leptospira spp
Parainfluenza
Pasteurella haemolytica
Pasteurella multocida
PI3
Tetanus Antitoxin
Tetanus Toxoid
Trichomonas
CANINE
Bordetella
Borrelia burgdorferi
CAV-2
Coronavirus
Distemper
Leptospira spp
Parainfluenza
Parvovirus
Rabies
Bordetella
Borrelia burgdorferi
CAV-2
Coronavirus
Distemper
Leptospira spp
Parainfluenza
Parvovirus
Rabies
EQUINE
Ehrlichia risticii
Encephalomyelitis
  Eastern
  Western
  Venezuelan
Influenza
Rabies
Rhinopneumonitis
Rotavirus
Streptococcus spp
Tetanus Antitoxin
Tetanus Toxoid
Viral arteritis
FELINE
Calicivirus
Chlamydia
Leukemia
Microsporum canis
Panleukopenia
Rabies
Rhinotracheitis
PORCINE
A pleuropneumoniae
Bordetella
E coli
Erysipelas
Haemophilus parasuis
Leptospira spp
Mycoplasma
Parvovirus
Pasteurella multocida
Pseudorabies
Tetanus Antitoxin
Tetanus Toxoid
```

In preferred embodiments antigens for veterinary applications are selected for use against rabies virus, canine parvovirus (severe gastrointestinal illness), canine parvovirus 2 (severe gastroenteritis), canine corona virus (gastroenteritis), canine adenovirus type 1(infectious hepatitis) and canine adenovirus type 2 (kennel cough), canine parainfluenza virus (tracheobronchitis, kennel cough), and numerous other animals pathogens.

The results of our studies indicate that molecular genetic manipulation of CDV is feasible and that rational design of future attenuated CDV strains and CDV expression vectors can be approached using cDNA rescue technology.

The rescue of rCDV provides one avenue to pursue development of safer live, attenuated immunogenic compositions for canine distemper virus. A further attenuated virus would be ideal if it remained effective for immunization of dogs and was safe and effective for use in other animals such as large cats, small carnivores and seals. For embodiments employing attenuated canine distemper viruses, conventional means are used to introduce attenuating mutations to generate a modified virus, such as chemical mutagenesis during virus growth in cell cultures to which a chemical mutagen has been added, followed by selection of virus that has been subjected to passage at suboptimal temperature in order to select temperature sensitive and/or cold adapted mutations, identification of mutant viruses that produce small plaques in cell culture, and passage through heterologous hosts to select for host range mutations. An alternative means of introducing attenuating mutations comprises making predetermined mutations using site-directed mutagenesis. One or more mutations may be introduced. These viruses are then screened for attenuation of their biological activity in an animal model. Attenuated canine distemper viruses are subjected to nucleotide sequencing to locate the sites of attenuating mutations.

Another approach to achieving this goal is to use a rational vaccine design strategy. There have been a number of studies that may help identify attenuating amino acid substitutions and cis-acting signal changes that could be tested in canine distemper virus. For example, studies of recombinant strains of human parainfluenza virus type 3 and respiratory syncytial virus have identified a number of attenuating mutations that may have good correlates in CDV. These include amino acid substitutions in the L protein (49, 50, 60), and mutations in cis-acting sequences in the leader and in GE/GS signals (21, 50, 59). In addition, the genome sequence of measles virus vaccines have been examined and compared to a wild-type isolate. There are examples of viruses defective for C or V protein expression that exhibit some degree of attenuation (12, 13, 15, 22, 31, 37, 53, 56). Specifically, one can insert into the CDV genome one or mutations that correspond to an attenuating mutation in a coding or non-coding region of another non-segmented, negative-sense, single stranded RNA Viruses of the Order Mononegavirales, and preferably, a virus from the Family Paromyxoviridae, such a PIV, RSV, Mumps and Measles. Various mutations for other viruses are well known and continue to be generated. Mutations which have been identified as attenuating for viruses of the Order Mononegavirales include, but are not limited to, the following: measles virus 3′ genomic promoter plus RNA polymerase gene (WO 98/13501), measles virus N, P and C genes, and F gene-end signal (WO 99/49017), respiratory syncytial virus 3′ genomic promoter plus RNA polymerase gene (WO 98/13501), respiratory syncytial virus M gene-end signal (WO 99/49017), respiratory syncytial virus RNA polymerase gene (U.S. Pat. No. 5,993,824), respiratory syncytial virus N and F genes (WO 00/61611), and parainfluenza virus type 3 3′ genornic promoter plus RNA polymerase gene (WO 98/13501). Once the mutation is made with the CDV genome, one can use the method of this invention to recombinantly-produced the recombinant virus. Futhermore, a gene inactivation approach may be useful. Finally, it may be possible to utilize the novel gene shuffling approach (3, 58) to develop a safer more attenuated strain of canine distemper virus for use in immunogenic and pharmaceutical compositions.

A rescued recombinant canine distemper virus is tested for its desired phenotype (temperature sensitivity, cold adaptation, plaque morphology, and transcription and replication attenuation), first by in vitro means, such as sequence identification, confirmation of sequence tags, and antibody-based assays. If the attenuated phenotype of the rescued virus is present, challenge experiments can be conducted with an appropriate animal model or target animal. These animals are first immunized with the attenuated, recombinantly-produced virus, then challenged with the wild-type form of the virus. The level of attenuation of the recombinantly-produced CDV is established by comparing the virulence of the attenuated virus to that of a wild type CDV or other standard (e.g. an accepted attenuated form of CDV). Preferably, the comparison establishes that an attenuated recombinant virus exhibits substantial reduction in virulence over the wild type. The level of virulence for the attenuated recombinant virus should be sufficient to permit using the recombinant virus in treating humans or in treating a select class of non-human animals.

The choice of expression vector as well as the isolated nucleic acid molecule which encodes the trans-acting proteins necessary for encapsidation, transcription and replication can vary depending on the selection of the desired virus. The expression vectors are prepared in order to permit their co-expression with the transcription vector(s) in the host cell and the production of the recombinant virus under selected conditions.

A canine distemper rescue includes an appropriate cell milieu, in which T7 RNA polymerase is present to drive transcription of the antigenomic (or genomic) single-stranded RNA from the viral genomic cDNA-containing transcription vector. Either co-transcriptionally or shortly thereafter, this viral antigenome (or genome) RNA transcript is encapsidated into functional templates by the nucleocapsid protein and engaged by the required polymerase components produced concurrently from co-transfected expression plasmids encoding the required virus-specific trans-acting proteins. These events and processes lead to the prerequisite transcription of viral mRNAs, the replication and amplification of new genomes and, thereby, the production of novel viral progeny, i.e., rescue.

In the rescue method of this invention, a T7 RNA polymerase can be provided by recombinant vaccinia virus. This system, however, requires that the rescued virus be separated from the vaccinia virus by physical or biochemical means or by repeated passaging in cells or tissues that are not a good host for poxvirus. This requirement is avoided by using as a host cell restricted strain of vaccinia virus (e.g. MVA-T7) which does not proliferate in mammalian cells. Two recombinant MVAs expressing the bacteriophage T7 RNA polymerase have been reported. The MVA/T7 recombinant viruses contain one integrated copy of the T7 RNA polymerase under the regulation of either the 7.5K weak early/late promoter (Sutter et al., 1995) or the 11K strong late promoter (74).

The host cell, or cell line, that is employed in the transfection of the rescue composition can vary widely based on the conditions selected for rescue. The host cells are cultured under conditions that permit the co-expression of the vectors of the rescue composition so as to produce the desired recombinant canine distemper virus. Such host cells can be selected from a wide a variety of cells, including a eukaryotic cells, and preferably vertebrate cells. Avian cells may be used, but if desired host cells can be derived from other cells, even human cells, such as a human embryonic kidney cell. Exemplary host cells are human 293 cells, A549 cells (lung carcinoma) and Hep2 cells (cervical carcinoma). Vero cells (monkey kidney cells), as well as many other types of cells, can also be used as host cells. Other examples of suitable host cells are: (1) Human Diploid Primary Cell Lines: e.g. WI-38 and MRC5 cells; (2) Monkey Diploid Cell Line: e.g. FRhL—Fetal Rhesus Lung cells; (3) Quasi-Primary Continuous Cell Line: e.g. AGMK-African green monkey kidney cells.; (4) other potential cell lines, such as, CHO, MDCK (Madin-Darby Canine Kidney, DK (dog kidney) and primary chick embryo fibroblasts (CEF). Some eukaryotic cell lines are more suitable than others for propagating viruses and some cell lines do not work at all for some viruses. A cell line is employed that yields detectable cytopathic effect in order that rescue of viable virus may be easily detected. In the case of canine distemper, the transfected cells can be co-cultured on Vero cells because the virus spreads rapidly on Vero cells and makes easily detectable plaques. In general, a host cell which is permissive for growth of the selected virus is employed.

In alternatively preferred embodiments, a transfection-facilitating reagent may be added to increase DNA uptake by cells. Many of these reagents are known in the art. LIPOFECTACE (Life Technologies, Gaithersburg, Md.) and EFFECTENE (Qiagen, Valencia, Calif.) are common examples. Lipofectace and Effectene are both cationic lipids. They both coat DNA and enhance DNA uptake by cells. Lipofectace forms a liposome that surrounds the DNA while Effectene coats the DNA but does not form a liposome.

The transcription vector and expression vector can be plasmid vectors designed for expression in the host cell. The expression vector which comprises at least one isolated nucleic acid molecule encoding the trans-acting proteins necessary for encapsidation, transcription and replication may express these proteins from the same expression vector or at least two different vectors. These vectors are generally known from the basic rescue methods, and they need not be altered for use in the improved methods of this invention.

In the method of the present invention, a standard temperature range (about 32° C. to about 37° C.) for rescue can be employed; however, the rescue at an elevated temperature has been shown to improve recovery of the recombinant RNA virus. The elevated temperature is referred to as a heat shock temperature (See International Patent Publication Number WO 99/63064, published Dec. 9, 1999, which is hereby incorporated herein by reference). An effective heat shock temperature is a temperature above the standard temperature suggested for performing rescue of a recombinant virus at which the level of recovery of recombinant virus is improved. An exemplary list of temperature ranges is as follows: from 38° C. to about 47° C., with from about 42° C. to about 46° C. being the more preferred. Alternatively, it is noted that heat shock temperatures of 43° C., 44° C., and 45° C. are particularly preferred.

Numerous means are employed to determine the level of recovery of the desired recombinant canine distemper virus. As noted in the examples herein, a chloramphenicol acetyl transferase (CAT) reporter gene is used to monitor and optimize conditions for rescue of the recombinant virus. The corresponding activity of the reporter gene establishes the baseline and test level of expression of the recombinant virus. Other methods include detecting the number of plaques of recombinant virus obtained and verifying production of the rescued virus by sequencing.

In preferred embodiments, the transfected rescue composition, as present in the host cell(s), is subjected to a plaque expansion step (i.e. amplification step). The transfected rescue composition is transferred onto at least one layer of plaque expansion cells (PE cells). The recovery of recombinant virus from the transfected cells is improved by selecting a plaque expansion cell in which the canine distemper virus or the recombinant canine distemper virus exhibits enhanced growth. Preferably, the transfected cells containing the rescue composition are transferred onto a monolayer of substantially confluent PE cells. The various modifications for rescue techniques, including plaque expansion, are also set forth in International Patent Publication Number WO 99/63064, published Dec. 9, 1999.

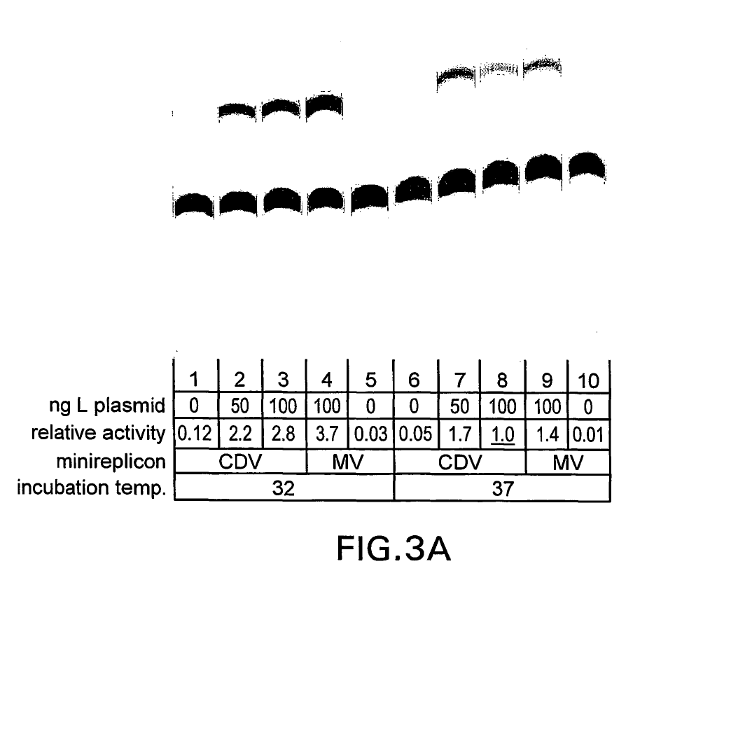

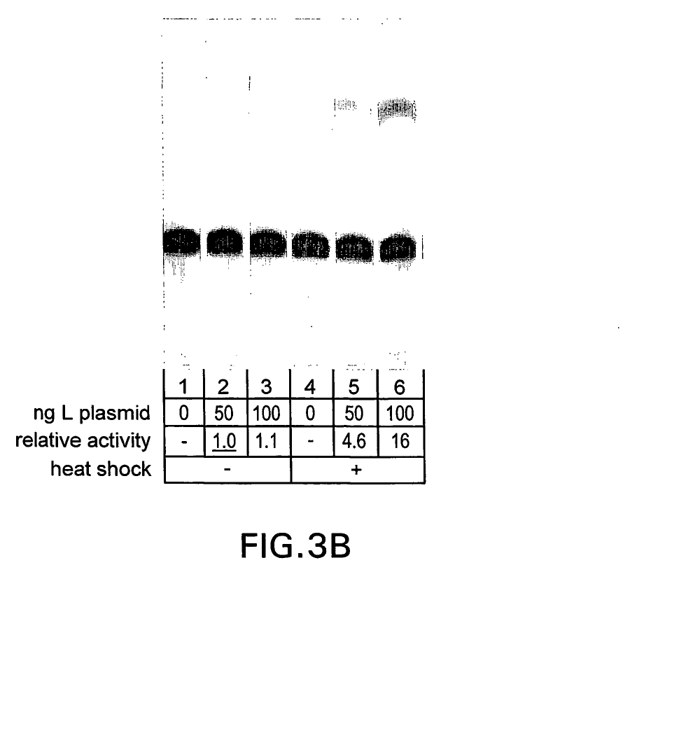

Additionally, it is noted that incubating the cells at temperatures between 30° C. to 35° C. rather than 37° C. increases minireplicon expression (see FIG. 3B). This observation has practical value for performing canine distemper virus rescue experiments at the lower temperature. Although lower incubation temperature increased minireplicon activity, it is found that transient incubation at elevated temperature increased CDV minireplicon activity. In view of the positive effect of heat shock on minireplicon activity, the heat shock step is preferably incorporated into our canine distemper virus rescue protocol of this invention.

Preferably, a rescue method employs a calcium-phosphate technique for method of transfection. The calcium-phosphate method generally increases the number of CDV-positive wells in a transfection experiments by about two-fold over the liposome method (data not shown). This can be important when isolating a highly attenuated strain. Without being bound by the following, calcium-phosphate may be less damaging to cell membranes than liposomal reagents and a healthier cell membrane promotes budding of relatively rare rescued virus. It could also be true that the calcium-phosphate precipitates are somewhat more effective at introducing multiple different plasmids into the same cell, and it actually generates more cells that contain the complete set of N, P, and L expression piasmids together with the genomic cDNA.

The preferred virus rescue method encompasses several of the aforementioned techniques, such as plaque expansion, heat shock, calicum precipitation techniques (10, 31, 40, 42), as well as several important modifications, such as low temperature incubation.

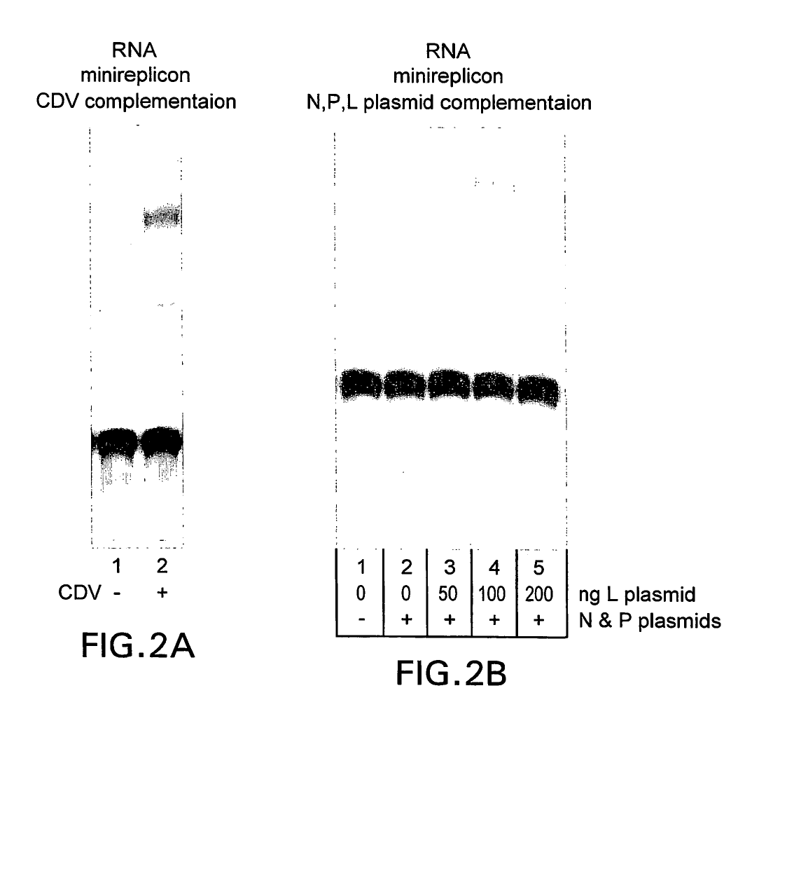

The varied combinations of techniques can be tested for optimizing the rescue method by using the minireplicon, which permits a rapid assessment a variety of variables that affect the levels of gene expression in a transient assay. For example, various components for rescue, including each expression vector (N, P, and L) as well as the cis-acting signals in the replicon vector, can be quickly tested to assess their activity within the rescue system. One can also use the minireplicon system to determine optimal amounts of expression vectors required for maximal minireplicon expression (See FIGS. 2 and 3; and data not shown). Each of these optimization steps produce beneficial increases in minireplicon expression and taken together they may have a significant effect on rescue. By combining two or more of the optimized variables and techniques, one can substantially improve the percentage of successfully rescued virus. The success rate can be measured by determining the number of positive wells per well plate. The success rate is at least about 50%, and even greater than 60%. In further preferred embodiments, the success rate is at least 75%, and more preferably, at least 80%. This is a substantial improvement when compared to published techniques for rescue (see for example, Published International Patent Application WO 99/63064).

For canine distemper virus, an optimized rescue method consistently generates 4-6 CDV-positive wells from a transfected six well plate using the modified protocol. In contrast, immunogenic compositions formed candidate strains, especially those containing desirable attenuating mutations, replicate very poorly and/or are difficult to rescue. The selected techniques for increased rescue efficiency may be applied for the rescue of any nonsegmented, negative-sense, single-stranded RNA virus. The current taxonomical classification of nonsegmented, negative-sense, single-stranded RNA virus, along with examples of each, is set forth below.

Classification of Nonsegmented, negative-sense, single stranded RNA Viruses of the Order Mononegavirales

Family Paramyxoviridae
Subfamily Paramyxovirinae
   Genus Respirovirus (formerly known as Paramyxovirus)
      Sendai virus (mouse parainfluenza virus type 1)
      Human parainfluenza virus (PIV) types 1 and 3
      Bovine parainfluenza virus (BPV) type 3
   Genus Rubulavirus
      Simian virus 5 (SV5) (Canine parainfluenza virus type 2)
      Mumps virus
      Newcastle disease virus (NDV) (avian Paramyxovirus 1)
      Human parainfluenza virus (PIV-types 2, 4a and 4b)
   Genus Morbillivirus
      Measles virus (MV)
      Dolphin Morbirivirus
      Canine distemper virus (CDV)
      Peste-des-petits-ruminants virus
      Phocine distemper virus
      Rinderpest virus
Subfamily Pneumovirinae
   Genus Pneumovirus
      Human respiratory syncytial virus (RSV)
      Bovine respiratory syncytial virus
      Pneumonia virus of mice
      Turkey rhinotracheitis virus

Family Rhabdoviridae
Genus Lyssavirus
   Rabies virus
Genus Vesiculovirus
   Vesicular stomatitis virus (VSV)
Genus Ephemerovirus
   Bovine ephemeral fever virus

Family Filovirdae
Genus Filovirus
   Marburg virus

To improve the efficiency of virus rescue for any of the above viruses, one varies the mass of N, P, and L expression vectors and mass of minireplicon of full length cDNA in order to generate amounts that enable one to rescue the recombinant virus. Thereafter, one can utilizes two or more of the following steps and/or techniques for increased rescue efficiency: (1) selecting the cell type for transfection (preferably, Vero cells, Hep2 or A549 cells); (2) selecting a transfection reagent (preferably, using a calcium phosphate reagent; (3) selecting an optimal cell type for conducting a plaque expansion step; and (4) selecting a cell type for that improves transfection. In addition, rescue efficiency is further improved by employing one or more of the following steps and/or techniques: (1) vary the incubation temperature on a given cell type and rescue system; (2) vary the timing of heat shock application (preferably, apply heat shock starting about 2 to about 4 hours after initiation of transfection); (3) vary the temperature of heat shock, (preferably about 42 to about 44° C.) and (4) vary the duration of heat shock (about 2 to about 3 hours is preferred). Additional increases of rescue efficiency are obtained also by selecting the appropriate amount of a T7 polymerase source, such as MVA/T7 or recombinant vaccina virus, and/or by adjusting the length of time cells that are exposed to a transfection reagent and DNAs in transfection.

The recombinant canine distemper viruses prepared from the methods of the present invention are employed for diagnostic, prophylactic and therapeutic applications. Preferably, the recombinant viruses prepared from the methods of the present invention are attenuated. The attenuated recombinant virus should exhibit a substantial reduction of virulence compared to the wild-type virus which infects human and animal hosts. The extent of attenuation is such that symptoms of infection will not arise in most individuals, but the virus will retain sufficient replication competence to be infectious and elicit the desired immune response profile for the desired immunogenic composition. The attenuated recombinant virus can be used alone or in conjunction with pharmaceuticals, antigens, immunizing agents or adjuvants, as immunogenic compositions in the prevention or amelioration of disease. These active agents can be formulated and delivered by conventional means, i.e. by using a diluent or pharmaceutically acceptable carrier.

Further embodiments of this invention an un-attenuated or attenuated recombinantly produced canine distemper virus is employed in immunogenic compositions comprising (i) at least one recombinantly produced canine distemper virus and (ii) at least one of a pharmaceutically acceptable buffer or diluent, adjuvant or carrier. Preferably, these compositions have therapeutic and prophylactic applications as immunogenic compositions in preventing and/or ameliorating canine distemper infection. In such applications, an immunologically effective amount of at least one recombinant canine distemper virus of this invention is employed in such amount to cause a substantial reduction in the course of the normal canine distemper infection.

The formulation of such immunogenic compositions is well known to persons skilled in this field. Immunogenic compositions of the invention may comprise additional antigenic components (e.g., polypeptide or fragment thereof or nucleic acid encoding an antigen or fragment thereof) and, preferably, include a pharmaceutically acceptable carrier. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The term “pharmaceutically acceptable carrier” refers to a carrier that does not cause an allergic reaction or other untoward effect in patients to whom it is administered. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antigen. The use of such media and agents for pharmaceutically active substances is well known in the art. Except insofar as any conventional media or agent is incompatible with the active ingredient, use thereof in the immunogenic compositions of the present invention is contemplated.

Administration of such immunogenic compositions may be by any conventional effective form, such as intranasally, parenterally, orally, or topically applied to mucosal surface such as intranasal, oral, eye, lung, vaginal, or rectal surface, such as by aerosol spray. The preferred means of administration is parenteral or intranasal.

Oral formulations include such normally employed excipients as, for example, pharmaceutical grades of mannitol, lactose, starch, magnesium stearate, sodium saccharine, cellulose, magnesium carbonate, and the like.

The immunogenic compositions of the invention can include an adjuvant, including, but not limited to aluminum hydroxide; aluminum phosphate; Stimulon™ QS-21 (Aquila Biopharmaceuticals, Inc., Framingham, Mass.); MPL™ (3-O-deacylated monophosphoryl lipid A; RIBI ImmunoChem Research, Hamilton, Mont.), IL-12 (Genetics Institute, Cambridge, Mass.); N-acetyl-muramyl-L-theronyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalrnitoyl-sn-glycero-3-hydroxyphos-phoryloxy)-ethylamine (CGP 19835A, referred to a MTP-PE); and cholera toxin. Others which may be used are non-toxic derivatives of cholera toxin, including its B subunit (for example, wherein glutamic acid at amino acid position 29 is replaced by another amino acid, preferably, a histidine in accordance Published Patent Application Number WO 00/18434, which is hereby incorporated herein), and/or conjugates or genetically engineered fusions of non-canine distemper polypeptides with cholera toxin or its B subunit, procholeragenoid, fungal polysaccharides.

The recombinantly-produced attenuated canine distemper virus of the present invention may be administered as the sole active immunogen in a immunogenic composition. Alternatively, however, the immunogenic composition may include other active immunogens, including other immunologically active antigens against other pathogenic species, as noted above. The other immunologically active antigens may be replicating agents or non-replicating agents. Other immunologically active antigens may be those directed against a variety of infectious agents (1, 7). The immuogenic compositions may used to treat a variety of animals, including companion animals, such as dogs (canine) and cats (feline), and also farm animals, such as bovine, swine and equine.

One of the important aspects of this invention relates to a method of inducing immune responses in a mammal comprising the step of providing to said mammal an immunogenic composition of this invention. The immunogenic composition is a composition which is immunogenic in the treated animal or human such that the immunologically effective amount of the polypeptide(s) contained in such composition brings about the desired response against canine distemper infection. Preferred embodiments relate to a method for the treatment, including amelioration, or prevention of canine distemper infection in an animal comprising administering to an animal an immunologically effective amount of the antigenic composition. The dosage amount can vary depending upon specific conditions of the individual. This amount can be determined in routine trials by means known to those skilled in the art. Animals and even humans can be treated with the immunogenic compositions of this invention. Certainly, a wide variety of animals may be treated. Animals for treatment include companion animals such as pet dogs as well as wild animals, such as foxes, wolves and coyotes. Since even red pandas have been reported as susceptible to infection by canine distemper virus, one might treat any animals that is in a contained area or environment, such those in zoos or wildlife parks. A canine distemper virus outbreak has been reported for seals and carnivores like mink, ferrets and raccoon, any of which may be a target animal for treatment as described hereinabove.

The following examples are included to illustrate certain embodiments of the invention. However, those of skill in the art should, in the light of the present disclosure, appreciate that many changes can be made in the specific embodiments which are disclosed and still obtain a like or similar result without departing from the spirit and scope of the invention.

### EXAMPLES

### Example 1

Materials and Methods

Cells and viruses. HEp2, A549, Vero, B95-8, and chicken embyro fibroblasts (CEF) cells were maintained in Dulbecco's modified Eagle medium (DMEM) supplemented with 10% fetal bovine serum (FBS). HeLa suspension cells were grown in modified minimal essential media (SMEM) supplemented with 5% FBS. The laboratory-adapted Onderstepoort CDV strain (17) was propagated in HeLa cells as described previously (46). A second Laboratory adapted Onderstepoort strain was provided by Dr. Martin Billeter of the University of Zurich and was propagated in B95-8 cells. The recombinant attenuated vaccinia virus strain MVA/T7 (obtained from Dr. B. Moss, National Institutes of Health, Bethesda, Md.; see Wyatt et al., 1995; ref.61) designed to express the T7 RNA polymerase gene was propagated in CEF cells. Stocks of MVA/T7 were titered on CEFs. The laboratory-adapted Edmonston strain of measles virus (MV) was grown in HeLa suspension cells (55).

Recombinant DNA.

Molecular cloning procedures were performed following standard protocols (2, 26, 71).

1A—Full-Length CDV cDNA Clone

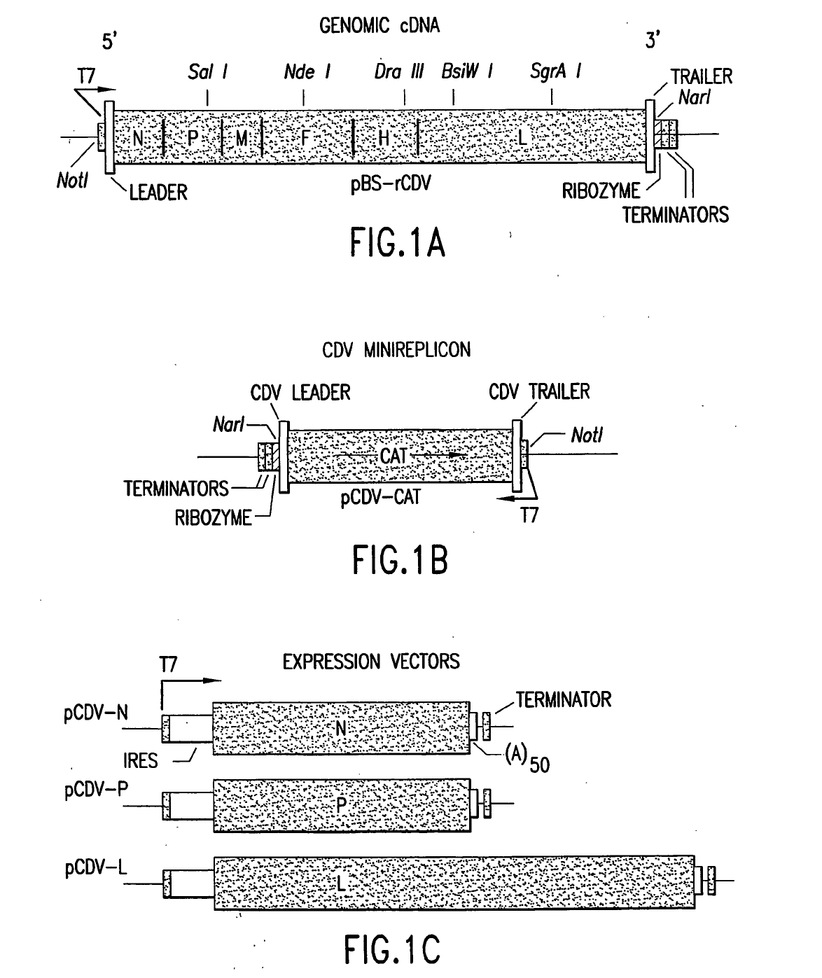

The full-length CDV cDNA clone was assembled from six RT/PCR fragments that take advantage of convenient restriction sites found in the genome (FIG. 1A). The viral cDNA was cloned with a T7 RNA polymerase promoter fused to the 5′ end of the positive genome strand and the 3′ end was flanked by the hepatitis delta virus ribozyme and two T7 transcriptional terminators. The T7 RNA polymerase promoter was truncated at the 3′ end by removal of the three G residues that normally provide the preferred T7 polymerase transcription initiation site so that a significant portion of the transcripts would initiate at the first A residue in the positive genome strand.

A plasmid vector containing unique NotI and NarI sites was prepared to facilitate cloning of the CDV full-length genomic clone. NotI and NarI restriction sites are absent in the CDV genome making them convenient sites for use in the vector backbone. This modified vector DNA was generated by PCR. Primers were designed to amplify the vector backbone from the previously reported measles virus minireplicon plasmid (FIG. 1, p802, ref 45). These primers directed amplification of the vector backbone and excluded the measles virus minireplicon sequences. The amplified DNA maintained the NarI site located in the ribozyme sequence and created a NotI site (see NotI and NarI site in FIG. 1A). The primers also contained 5′ extensions designed to generate a polylinker between the NotI and NarI sites once the amplified DNA was ligated to circularize the amplified vector backbone for bacterial transformation. The polylinker contained SalI, NdeI, DraIII, BsiWI and SgrA1 sites to facilitate cloning fragments amplified from the viral genome (FIG. 1A).

The full-length genomic cDNA was cloned in the vector described above (FIG. 1a). The completed CDV cDNA sequence was 15,690 bases, a number divisible by six, in agreement with the rule-of-six (6, 23). The viral cDNA in plasmid pBS-rCDV (FIG. 1A) was oriented to permit synthesis of a positive-sense copy of the CDV genome by T7 RNA polymerase. To prepare the genomic cDNA plasmid, six fragments of the CDV genome (FIG. 1A) were sequentially cloned after reverse transcription and PCR amplification (RT/PCR) from purified viral RNA (46).

The first genomic cDNA fragment amplified was equivalent to the NarI/SgrAI fragment in FIG. 1A (CDV nucleotides 13089-15690 of SEQ ID NO.2). The primer used for amplifying the 3′ end of the CDV cDNA was complementary to the CDV terminus and contained an extension that included ribozyme sequence spanning the NarI site (cagccggcgccagcgaggaggctgggaccatgccggccACCAGACAAA GCTGGGT, SEQ ID NO. 6, in which CDV sequence is capitalized). The second primer spanned the SgrAI site in the viral genome (TACTCAAGTCAAATACTCAGGGAC, SEQ ID NO. 7). The amplified fragment was digested with NarI and SgrAI and cloned into the vector backbone. This plasmid was then used to clone in the next fragment that spanned the SgrAI and BsiWI sites(10136-13088; primers CAGGGGTGCTTTTCTGAGTCACTGC, SEQ ID NO. 8 and ACGACCTTTCTGAGCCCTGGGACTC, SEQ ID NO. 9). Similarly, the BsiWI-DraIII fragment (nucleotides 8666-13015; primers AGAGGAGACCAGTTCACTGTACTCC, SEQ ID NO. 10 and TGATTCCCTCCCCTGAGGCATGAGC, SEQ ID NO. 11), the NdeI/DraIII fragment (nucleotides 5845-8665; primers GCAATCCAATCTCTTAGAACCAGCC, SEQ ID NO. 12 and TCGAATCTGTAAAATTGGTGACACC, SEQ ID NO. 13) and the SalI/NdeI fragment (nucleotides 2962-5844; primers GCCATTACTAAACTAACTG, SEQ ID NO. 14 and ATCTTATGAATTTCTCCTCC, SEQ ID NO. 15) were amplified and sequentially added to the growing cDNA clone. Finally, the NotI/SalI fragment containing the T7 promoter plus CDV nucleotides 1-2961was amplified (primers ATGGGTTTCAGCTGGAGGTCTCTC, SEQ ID NO. 16 and cggcggccgcgtaatacgactcactata ACCAGACAAAGTTGGCT, SEQ ID NO. 17, in which CDV nucleotides capitalized) and added to genomic cDNA clone.

The completed genomic cDNA plasmid was sequenced and compared to the CDV genomic consensus sequence. This revealed a number of nucleotide changes that were most likely introduced by RT/PCR amplification. Some base changes in protein coding regions were silent with respect to amino acid codon specificity. These base substitutions were not repaired; They served as useful “tags” to identify a recombinant virus. In addition, one noncoding region base change was also found in the intergenic region between the M and F genes (M/F intergenic region) at nucleotide 6837 and this base substitution was not repaired. Base substitutions that affected codon specificity were repaired by oligonucleotide mutagenesis or by replacement of a mutated region with an independently RT/PCR-amplified DNA fragment. Oligonucleotide mutagenesis was performed by first subcloning the region that required base correction then using either the QuickChange (Stratagene) or Morph (5 prime-3 prime, Inc) mutagenesis kits to make the correction. The corrected fragment was then shuttled back into the full-length clone. The repaired full-length clone was sequenced to confirm correction of mutations.

1B—CDV Minireplicon

The plasmid vector used for the full-length cDNA clone was also used to generate pCDV-CAT containing CDV minireplicon (CDV-CAT) sequences. The sequences that compose the CDV minireplicon include the CAT gene flanked by the CDV leader at the 5′ end of the reporter gene and the CDV trailer at the 3′ end (FIG. 1B). The CDV minireplicon was inserted between the T7 polymerase promoter and ribozyme in the opposite direction of the full-length clone. Thus, T7 RNA polymerase transcription generates the equivalent of a negative-strand minigenome RNA.

The CDV minireplicon was cloned into the vector backbone described above. Minireplicon DNA used for cloning was prepared by PCR. The CAT gene flanked by the CDV leader and ribozyme sequence (including the NarI site) at the 5′ end, and the CDV trailer and T7 promoter at the 3′ end (FIG. 1B), was generated by four nested PCR reactions. Briefly, the CAT gene was amplified four different times using four different sets of primers. The first set of primers targeted amplification of the CAT gene while adding parts of the leader and trailer to the PCR product by virtue of sequences incorporated at the 5′ end of the CAT gene-specific primers. The next round of PCR used primers that overlapped the first set of primers while adding additional sequences from the CDV leader and trailer. This scheme of using overlapping primers with 5′ extensions was repeated four times using primers from the list below:

| | |
|---|---|
| Plasmid minireplicon 5′ end primers | |
| A | |
| 65 CDV 107 CDV CAT gene 5′ end | |
| \| \| | |
| GATCCTACCTTAAAGAACAAGGCTAGGGTTCAGACCTACCAATATGGAGAAAAAAATCAC | SEQ ID NO. 18 |
| B | |
| 26 CDV CDV 85 | |
| \| \| | |
| TTAAATTATTGAATATTTTATTAAAAACTTAGGGTCAATGATCCTACCTTAAAGAACAAG | SEQ ID NO. 19 |
| C | |
| 1 CDV CDV 57 | |
| \| \| | |
| ACCAGACAAAGTTGGCTAAGGATAGTTAAATTATTGAATATTTTATTAAAAACTTAG | SEQ ID NO. 20 |
| D | |
| Ribozyme sequence CDV 1 CDV 24 | |
| NarI \| \| | |
| GGCGCCAGCGAGGAGGCTGGGACCATGCCGGCCACCAGACAAAGTTGGCTAAGGATA | SEQ ID NO. 21 |
| E | |
| Ribozyme sequence CDV 1 CDV 24 | |
| NarI \| \| | |
| ATTGGCGCCAGCGAGGAGGCTGGGACCATGCCGGCCACCAGACAAAGTTGGCTAAGGATA | SEQ ID NO. 22 |
| Plasmid minireplicon 3′ end primers | |
| F | |
| 15624 CDV CDV 15584 CAT gene 3′ end | |
| \| \| | |
| TAGCAATGAATGGAAGGGGGCTAGGAGCCAGACTAACCTGTCATTACGCCCCGCCCTGC | SEQ ID NO. 23 |
| stop codon from CDV L gene ••• *** stop codon from CAT | |
| G | |
| 15666 CDV CDV 15608 | |
| \| \| | |
| ACTTATTAATAACCGTTGTTTTTTTTCGTATAACTAAGTTCAATAGCAATGAATGGAAGG | SEQ ID NO. 24 |
| H 15690 CDV CDV 15640 | |
| \| \| | |
| CTCACTATAACCAGACAAAGCTGGGTATGATAACTTATTAATAACCGTTGTTTTTTTTCG | SEQ ID NO. 25 |
| I 15690 CDV CDV 15662 | |
| HindIII T7 promoter \| \| | |
| ATTGCGGCCGCTAATACGACTCACTATAGGGACCAGACAAAGCTGGGTATGATAACTTAT | SEQ ID NO. 26 |
| J 15690CDV CDV 15661 | |
| NotI T7 promoter \| \| | |
| ATTGCGGCCGCTAATACGACTCACTATAGGGACCAGACAAAGCTGGGTATGATAACTTAT | SEQ ID NO. 27 |

Four rounds of nested PCR amplification generated the minireplicon fragment consisting of: 5′—NarI site—33 bp of ribozyme sequence—CDV leader—CAT gene—CDV trailer—T7 promoter—HindIII site. The four nested PCR amplifications were performed with the following primer pairs: Round 1: Primers A+F; Round 2: Primers B+G; Round 3: Primers C+H; Round 4: Primers D+I.

One may notice that primer F specified two stop codons at the 3′ end of the CAT gene. One was the CAT gene stop codon and the other was derived from the L gene in the CDV genome. Two stop codons were incorporated simply to introduce 3 additional nucleotides (the second stop codon) to make the minigenome comply with the rule-of-six (6, 23).

At this point, the initial plan was to use a vector that contained a HindIII site at the location of the NotI site in FIG. 1B. Accordingly, primer I listed above contained a HindIII site. The decision to use a NotI site in the vector led to a fifth round of PCR to generate a minireplicon fragment containing a NotI site. To introduce the NotI site, the minireplicon DNA was amplified with primers E and J (J contains the NotI site).

Finally, the primers used above incorporated a wild-type T7 promoter sequence (TAATACGACTCACTATAGGG, SEQ ID NO. 28, see primers H, I, and J) in the CDV minireplicon. Poor minireplicon activity in transfection experiments led to further modification of the minireplicon to remove the three G residues (italics) from the 3′ end of the T7 promoter. These residues in the T7 promoter are actually copied by the polymerase and incorporated into the minireplicon transcript. This generates a minireplicon RNA that does not comply with the rule-of-six (6, 23). Truncation of the T7 promoter reduces promoter activity but generates a minireplicon transcript that follows the rule-of-six. The modified minireplicon was generated by PCR amplification using primers similar to E and J with modified primer J lacking the three G residues.

1C—CDV Construct for Expressing Heterologous Nucleic Acid or Gene Sequences.

The genomic cDNA clone was modified between the P and M genes to permit insertion of foreign genes. Modifications were selected to allow introduction of several unique restriction sites while minimally modifying the CDV sequence. Eight nucleotide substitutions were introduced creating three unique restrictions sites (3330 G to A, 3331 G to A, 3335 T to C, 3348 A to G, 3349 A to G, 3355, G to C, 3373 T to A, and 3377 T to G). These modifications created three unique restriction sites (AatII, FseI and MluI, FIG. 5A) between CDV nucleotide positions 3329 to 3377. A ninth base change was added (3337, A to T) just 3′ of the AatII site to knockout a SalI site that was generated by the nucleotide changes used to generate the AatII site.

The nucleotide substitutions were first created in a plasmid subclone (SalI position 2961 to NdeI position 5843) that contained the P and M intergenic region. The modified fragment from the subclone was swapped back into the full-length genomic clone positioning the new restriction enzyme sites 3′ of the P gene open reading frame and 5′ of the P/M gene-end/gene-start signal. This clone (pBS-rCDV+) was rescued successfully demonstrating that the base substitutions did not have a noticeable deleterious effect on the virus.

Clone pBS-rCDV+ was then used as a vector for insertion of a foreign gene. The luciferase gene from pGL2-luc (Promega) was amplified with primers (5′ end, TACTGGCCGGCCATTATAAAAAACTT AGGACACAAGAGCCTAAGTCCGCTGCCACCATGGAAGACGCCAAAAA CAT, SEQ ID NO. 29; 3′ end, TTTTACGCGTTTAC AATTTGGACTTTCCGC, SEQ ID NO. 30) that incorporated a 5′ FseI and 3′ MluI site into the luciferase gene. The 5′ end primer, specific for the amino terminus of the luciferase coding region, also contained a 5′ extension that included a copy of the GE/GS signal from the P/M intergenic region in addition to the FseI site (FIG. 5A). The primers used to amplify the luciferase gene were designed to produce a fragment that took into account the rule-of-six (23) when it was finally inserted into pBS-rCDV+ to generate pBS-rCDV-P/luc/M (FIG. 5A).

1D—Expression Vectors pCDV-N, pCDV-P, and pCDV-L Expression vectors pCDV-N, pCDV-P, and pCDV-L were prepared by inserting the N (nucleotides 108-1679), P (1801-3324), or L (9029-15584) coding sequences into a vector based on pTM-1 (29, 41) as shown in FIG. 1C. This vector contains the T7 RNA polymerase promoter upstream of the encephalomyocarditis virus internal ribosome entry site (IRES). A NcoI site located at the 3′ end of the IRES is used for cloning and also contains the ATG initiator codon. A synthetic polyadenosine stretch is located just 3′ of the cloning region followed by a T7 RNA polymerase terminator. The N, P and L gene inserts were prepared by PCR amplification. The N and P genes were amplified from infected-cell RNA by RT/PCR. The L gene was PCR-amplified from the full-length CDV cDNA clone. Errors introduced during PCR were repaired by replacing mutated sequences with fragments generated from an independent PCR amplification or by oligonucleotide-directed mutagenesis. The MV N, P and L genes from the laboratory-adapted Edmonston strain (55) were cloned into the T7 vector after RT/PCR amplification from infected cell RNA.

1E—DNA Sequencing and Sequence Confirmation.

The sequence of the genes cloned in expression vectors, the sequence of pCDV-CAT, and the sequence of full-length genomic clones were determined by cycle-sequencing (16, 24) using dye-terminator/Taq DNA polymerase kits (ABI). Sequencing reactions were purified on microspin G50 columns (Amersham-Pharmacia Biotech) and analyzed on an ABI 377 automated sequencer (ABI). Sequence data was analyzed by computer analysis with MacVector (Oxford Molecular).

The genomic sequence of the CDV Onderstepoort strain was confirmed by generating a consensus sequence directly from amplified RT/PCR products (36). Briefly, RNA from infected cells was extracted by the guanidinium-phenol-chloroform extraction procedure (9) using Trizol reagent (Life Technologies). Purified RNA was reverse-transcribed using gene-specific primers and Superscript II reverse transcriptase (Life Technologies). Gene-specific primers and Taq DNA polymerase (ABI) were then used to amplify genome fragments that were subsequently gel-purified. Purified PCR fragments were cycle-sequenced and analyzed as described above.

Authentication of Rescued CDV

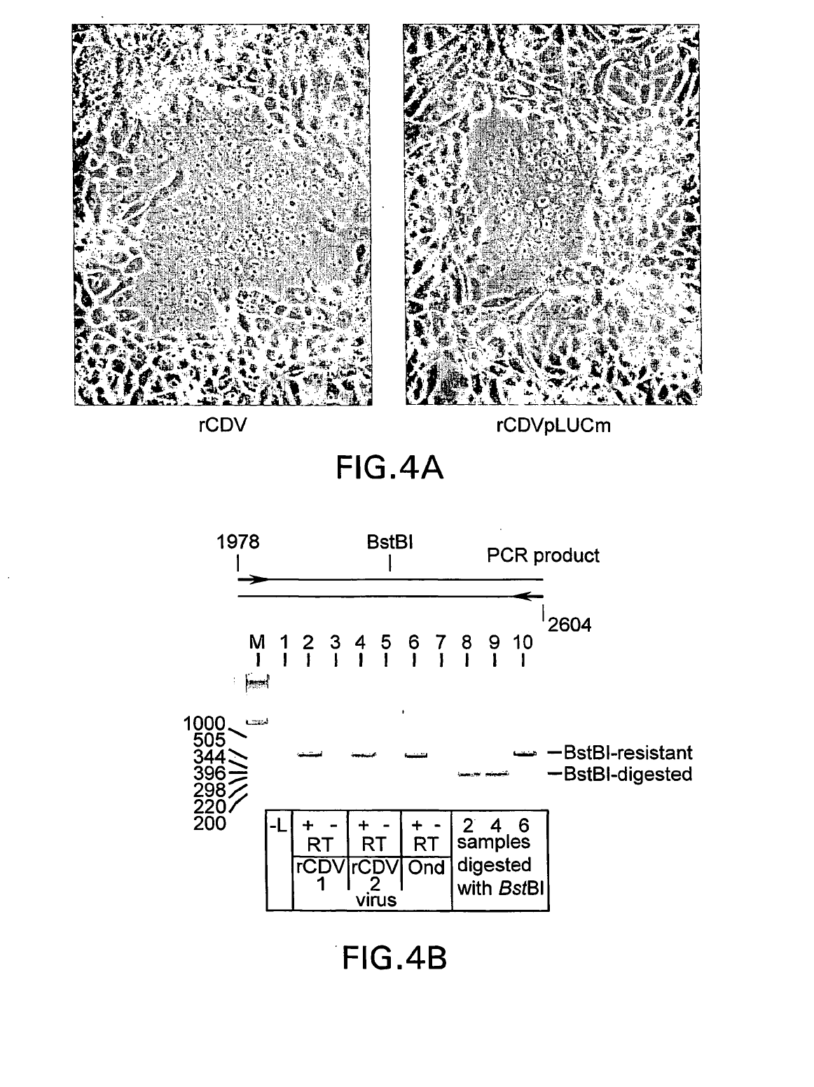

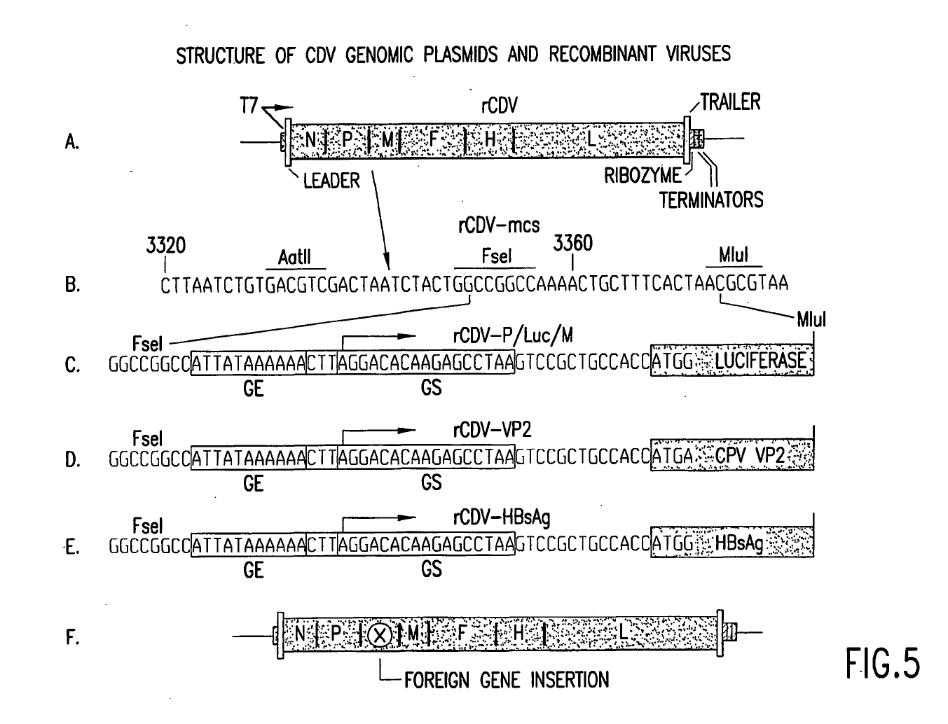

Sequence tags in the genomes of recombinant CDV (rCDV) isolates were analyzed by DNA sequencing or analyzed for the presence of restriction enzyme site markers. Fourteen nucleotide positions were used to distinguish between rCDV and CDV strains used in the laboratory. Infected-cell RNA was isolated by the guanidinium-phenol-chloroform extraction method as described above. The genomic region containing the appropriate sequence tag was amplified by RT/PCR using the Titan one-tube PCR kit (Roche Molecular Biology). Negative controls (−RT) that test for the presence of contaminating plasmid DNA were performed by adding RNA after the RT step was completed in the one-tube reaction system. PCR fragments were sequenced as described above, or the amplified fragment was digested with an appropriate restriction enzyme (FIG. 4B).

The rCDV genome containing the luciferase gene (rCDV-P/luc/M) was analyzed by sequence analysis to verify that the luciferase gene was correctly inserted. Cells infected with rCDV-P/luc/M isolates were also analyzed for luciferase expression. Infected cells extracts were prepared with Reporter Lysis Buffer (Promega: Madison, Wis.) and analyzed for luciferase activity using reagents from Pharmingen and an Analytical Luminescence Laboratories luminometer (Pharmingen, San Diego, Calif.).

### Example 2

General Methods for Transient Expression Analysis by CAT Assay and Virus Rescue

Minireplicon transfections were performed by several methods. For experiments in which the CDV minireplicon was transfected as RNA, 293 cells were transfected with Lipofectace (Life Technologies). Minireplicon RNA was prepared in vitro with T7 RNA polymerase (2) using pCDV-CAT DNA (FIG. 1B) as transcription template. The RNA was synthesized and purified using reagents and protocols in the Megascript kit (Ambion). In minireplicon experiments in which CDV infection provided complementation (FIG. 2A), the components of the RNA transfection mixture was prepared in two tubes. One tube contained 20 μg of purified minireplicon RNA and 100 μl serum-free OptiMEM (Life Technologies). The second tube was prepared with 100 μl of serum-free OptiMEM and 9-12 μl of Lipofectace (Life Technologies). The contents of both tubes were then mixed and allowed to incubate 30-40 min at room temperature. Before transfection, the culture media was removed from the 293 cell monolayers (approximately 80% confluent in a 60 mm dish) and the cells were washed once with serum-free OptiMEM. The RNA transfection mixture was then mixed with 0.8 ml of serum-free OptiMEM containing enough CDV (Ondestepoort) to infect the monolayer at a multiplicity of infection (moi) of approximately 2 plaque-forming units (pfu) per cell. This lml transfection mix was then added to cell monolayer and incubated at 37° C. for 5 hours. Following the 5 h incubation, 1 ml of DMEM supplemented with 20% FBS was added to the cells and incubation was continued overnight. Cell extracts were prepared at about 24 hours after transfection when greater than 70% of the cell monolayer exhibited cell fusion. CAT assays were performed basically as described previously (35). In some experiments (FIG. 3), C¹⁴-label chloramphenicol substrate was substituted with a fluorescent substrate (20, 62) and the assays were modified according to the substrate manufacturer's protocol (FAST CAT Yellow or Fast CAT Green; Molecular Probes). Products of fluorescent CAT assays were analyzed on a FlourImager (Molecular Dynamics) and quantitated using ImageQuant software (Molecular Dynamics).

RNA minigenome was also cotransfected with N, P and L expression plasmids. The transfection was performed essentially as described above except that the RNA was combined with the appropriate plasmid DNAs (1 μg pCDV-N and pCDV-P, 200 ng pCDV-L), 100 μl serum-free OptiMEM, and 20 μl of Lipofectace (Life Technologies). One hour prior to transfection the 293 cell monolayer was infected with MVA/T7 at an moi of five pfu per cell to provide T7 RNA polymerase to transcribe the expression plasmids.

Transfection protocols described above were modified for DNA minireplicon transfections and followed a protocol similar to described by Whitehead et al. (59). In these experiments we switched from 293 cells to HEp2 or A549 cells because we found that they were noticeably more resistant to the effects of MVA/T7 infection. The cells used for transfection were normally about 70-90% confluent. Transfection mixes were prepared by combining minireplicon DNA (10 ng pCDV-CAT) and expression plasmids (400 ng pCDV-N, 300 ng pCDV-P, 50-100 ng pCDV-L) in 200 μl of serum-free OptiMEM before adding 15 μl of Lipofectace (Life Technologies). This mixture was incubated 20 to 30 min at room temperature. A separate MVA/T7 mixture was prepared in sufficient quantity to provide 0.8 ml of serum-free OptiMEM containing enough MVA/T7 to infect each well of a six-well plate with about 2-5 pfu per cell. Before initiating the transfection, the culture media was removed from the monolayer and the transfection mix was added to 800 μl of the MVA/T7 mix and the combined 1 ml mixture was added to the cells. After overnight incubation, the transfection media was replaced with DMEM supplemented with 10% FBS and the cells were incubated an additional day. About 48 hours after the start of transfection, the cells were harvested and extracts prepared for analysis of CAT activity as described above. As indicated in the legends, some Iinireplicon experiments (FIG. 3B) were performed using the calcium-phosphate transfection procedure essentially as described below for virus rescue.

Transfection of cells for virus rescue was performed primarily with a calcium-phosphate method. We also used the Lipofectace protocol described above but found that the calcium-phosphate procedure combined with a heat shock step (35) was more effective. A549 cells or HEp2 monolayers in six-well plates were 75-90% confluent before transfection. 1-2 hours before transfection, the cells were fed with 4.5 ml of DMEM containing 10% FBS and shifted to an incubator set at 3% CO₂. Normally, this incubator was also set to 32° C. rather then 37° C., since minireplicon experiments indicated that this lower temperature would likely yield greater levels of rescue (FIG. 3A). The calcium-phosphate-DNA precipitates were prepared by first combining full-length CDV plasmid (5 μg) with 400 ng pCDV-N, 300 ng pCDV-P, and 100 ng pCDV-L and adjusting the fmal volume to 225 μl with water in a 5 ml polypropylene tube. Next, 25 μl of 2.5M calcium chloride was added to the DNA solution. Finally, 250 μl of 2×BES-buffered saline (50 mM BES [pH 6.95-6.98], 1.5 mM NaHPO₄, 280 mM NaCl) was added drop-wise to the tube while gently vortexing the mixture. The precipitate was allowed to form for 20-30 min at room temperature. The precipitate was then added drop-wise to the culture media followed by addition of sufficient MVA/T7 to provide an MOI of 1-3. The plate was rocked gently to ensure uniform mixing of the media, calcium-phosphate-DNA precipitate, and MVA/T7 before returning the cells to the incubator set at 3% C0₂. Three hours after starting the transfection, the six-well plate was sealed in a zip-lock plastic bag and submersed in a water bath set at 43-44° C. for 2 hours. After heat shock, the cells were returned to the 32° C. incubator set at 3% CO₂. The following day, the transfection media was removed and the cells were washed with a hepes-buffered saline solution (10 mM hepes [pH 7.0], 150 mM NaCl, 1 mM MgCl₂) and fed with 2-3 ml of DMEM supplemented with 10% FBS. The cells were incubated an additional 24-48 hours at 32° C. At 48-72 hrs after initiation of transfection, the cells were scraped into the media and transferred to a 10 cm plate containing a 70-80% confluent monolayer of Vero cells and 10 ml of media to initiate a coculture (35). At 3-5 hours after starting the coculture, the media was replaced with 10 ml of DMEM containing 10% FBS. Four to six days later, plaques were evident. Rescued virus was harvested for later analysis by scraping the cells into the media and freezing at −80° C.

### Example 3

CDV minireplicon expression. Transient expression studies using a minireplicon reporter system are important for developing a virus rescue system. Analyzing transient expression from a minireplicon reporter permits relatively rapid evaluation of transfection parameters to determine optimal conditions, and also is a valuable tool to determine whether expression vectors for N, P and L direct synthesis of functional proteins.

3.1 CDV Minireplicon Rescue by Virus

3.1.1 This minireplicon experiment tests whether the CDV-CAT minireplicon is functional by its ability to be rescued by virus complementation. CDV-CAT minireplicon RNA (20 μg) synthesized in vitro was transfected into 60 mm dishes of 293 cells. The cells were also infected with approximately CDV at an moi of approximately 2 when transfection was initiated. Approximately 24 hours after transfection, when about 70 percent of the cells were incorporated into syncytia, cell extracts were prepared and analyzed for CAT activity (FIG. 2A). Autoradiograms displaying the results of CAT assays are shown in FIG. 2A. CAT activity was readily detected in CDV-infected cells transfected with minireplicon RNA demonstrating that the minireplicon was functional (FIG. 2A, lane 2). Control cells that were transfected with RNA but not infected with CDV produced no detectable CAT activity (FIG. 2A, lane 1) demonstrating that the CAT activity was apparently due to replication and expression of the minireplicon.

3.1.2 After establishing that CDV minireplicon RNA was functional when provided with trans-acting proteins expressed from a complementing virus, we next tested the ability of the N, P and L protein expression vectors (FIG. 1C) to provide complementation. Accordingly, minireplicon RNA (20 μg) was cotransfected along with pCDV-N (1 μg), pCDV-P (1 μg), and pCDV-L (amount shown in FIG. 2B). One hour prior to transfection, the 293 cells used in this experiment were infected with MVA-T7 at an moi of 5 to provide T7 RNA polymerase required for expression of N, P and L proteins from the plasmid vectors. Analysis of extracts from transfected cells for CAT activity demonstrated that the expression plasmids effectively provided complementation (FIG. 2B). CAT activity indicative of minireplicon replication and expression was detectable when 50 and 100 ng of pCDV-L expression plasmid was used (FIG. 2B) and was maximal at 100 ng. More than 100 ng of L expression plasmid was inhibitory (FIG. 2B, lane 5). As expected, very little or no CAT activity was detected in negative control transfections that received only minireplicon RNA (FIG. 2B, lane 1) or received no L protein expression vector (FIG. 2B, lane 2).

3.1.3 Rescue of CDV Minireplicon DNA

The conditions used in the fmal minireplicon experiments more closely mimic the conditions used to rescue full-length virus since minireplicon plasmid DNA rather then RNA was transfected into cells along with the expression vectors for N, P and L. Thus, synthesis of replicon RNA is dependent upon intracellular transcription by T7 RNA polymerase. The results in FIG. 3A demonstrate that minireplicon activity was obtainable after transfection of minireplicon DNA. In addition, to test the possibility that the activity of the miinireplicon may display some temperature sensitivity, we incubated transfected cells at different temperatures (FIG. 3A). A549 cells in six-well plates were transfected and incubated at 32° C. or 37° C. Plasmid minireplicon pCDV-CAT (50 ng) was cotransfected into A549 with expression plasmids (400 ng pCDV-N, 300 ng pCDV-P, 50 or 100 ng pCDV-L) using a liposome transfection reagent. Similarly, the measles virus minireplicon (100 ng pMV107-CAT) was cotransfected with measles virus protein expression vectors (400 ng pMV-N, 300 ng pMV-P, 100 ng pMV-L). Simultaneous with transfection, the cells were infected with MVA/T7 at an moi of approximately 2. Cell extracts were prepared at approximately 48 hours after transfection and CAT activity was analyzed. In the experiments shown in this figure, the CAT assay was performed with a fluorescent chloramphenicol substrate and reaction products were quantified using a flourimager. Relative CAT activity in FIG. (3A) is expressed relative to the value given in lane 8.

The results shown in FIG. 3 clearly demonstrated significant levels of CDV-CAT minireplicon activity (see FIG. 3A, lanes 2 and 3) over a negative control transfection in which the pCDV-L DNA was omitted (see FIG. 3A, lane 1). There was a low but detectable background signal observed in the absence of pCDV-L vector probably results from a cryptic vaccinia virus promoter or cellular RNA polymerase II promoter in the CDV leader. A minireplicon vector that is identical except for the presence of the MV leader and trailer generates nearly undetectable background when using significantly greater amounts of Mv minireplicon (see FIG. 3A, lane 5,(35, 41). These results also showed that incubation at 32° C. rather than 37° C. generally produced 2-3 fold higher levels of CDV-CAT activity. Accordingly, a temperature of 32° was used for virus rescue.

In addition, these experiments (FIG. 3) were performed with A549 or HEp2 (A549 in FIG. 3; HEp2, data not shown) cells because we observed that these cells seemed to better tolerate infection with MVA/T7 then did 293 cells (FIG. 2B). For some of these experiments, also a liposome transfection protocol (FIG. 3A) was replaced with a calcium-phosphate procedure (FIG. 3B). Additional variables were examined and are described below.

3.1.4 Heat Shock Application for CDV Minireplicon

A549 cells in six-well plates were cotransfected with the pCDV-CAT minireplicon (10 ng) and expression vectors for N (400 ng), P (300 ng), and L (50-100 ng) using the calcium-phosphate procedure described in the Methods. At 3 hours after initiating transfection, the cells were shifted to 43° C. for 2 hours then returned to 32° C. overnight. The effects of heat shock on expression of the CDV minireplicon are shown in FIG. 3B. The heat shock treatment increased CDV-CAT activity by about 4-16 fold, indicating that this treatment would likely be beneficial for rescue of CDV.

### Example 4

4.1 Rescue of rCDV.

The transfection and culture conditions described above that produced the greatest levels of minireplicon activity were applied to rescue of CDV, i.e. A549 or Hep2 cells were transfected with full-length cDNA plasmnid and pCDV-N, pCDV-P, and pCDV-L expression vectors using the calcium-phosphate method (Methods). Three hours after initiation of transfection, the cells were heat shocked for 2 hours at 43-44° C. then returned to a 32° C. incubator. The following day, the media was replaced and the transfected cells were incubated for an additional day. To identify transfected cell cultures that produced virus and expand the small amounts of any rCDV, the transfected cells were cocultured with a fresh monolayer of Vero cells (Methods; ref. 35). Syncytia were observed after 4-6 days of coculture at 32° C., (see FIG. 4A).

In most experiments, our rescue conditions produced 4-6 rCDV-positive wells from a transfected 6 well plate that were detectable after coculturing with Vero cells. We also conducted a limited comparison of rescue efficiency when using calcium-phosphate or a liposomal transfection reagent. The calcium phosphate procedure described in the methods resulted in CDV-positive transfections about 2 fold more often than the liposome reagent (data not shown).

4.2 Characterization of Rescued Virus

RNA from cells infected with two isolates of rCDV (rCDV1 and rCDV2) or the Onderstepoort strain obtained from Martin Billeter (Ond) was used to amplify a DNA fragment from the P gene. RT/PCR-amplified fragments from recombinant strains contain a restriction enzyme digestion site “tag” for BstBI. Non-recombinant (Ond) strains lack this site. The CDV isolates from several experiments were characterized to confirm that a recombinant virus was rescued. Recombinant CDV should contain the nucleotide changes (sequence “tags”) introduced during cDNA cloning that were not repaired. For example, there were two closely positioned base changes in the P gene (nucleotides 2295 and 2298) that were silent with respect amino acid codon specificity but generated a BstBI restriction enzyme digestion site. This BstBI tag in the recombinant cDNA should be absent from the two CDV strains used in the laboratory (our lab-adapted Onderstepoort strain and a lab-adapted Onderstepoort strain provided by Martin Billeter, University of Zurich) that would potentially serve as a source of contaminating virus. For example, a region of the P gene (from position 1978 to 2804) was amplified from infected-cell RNA by RT/PCR and subsequently digested with BstBI. The results clearly showed that recombinant virus contained the BstBI tag while the non-recombinant strain did not (see FIG. 4B, compare lanes 2, 3, 6 to lanes 8, 9, 10). The PCR product derived from the recombinant virus was cleaved by BstBI producing a doublet that migrated faster than the DNA that was resistant to digestion (see FIG. 4B). These results were confirmed also by directly sequencing the PCR-amplified DNA fragment. Eleven additional sequence tags were analyzed similarly and the results conclusively showed that a recombinant strain of CDV was being produced by rescue. The possibility that the analysis of sequence tags was complicated by contaminating genomic cDNA carried over from transfected cells can be ruled out by two negative controls. RNA prepared from cells originating from a negative control transfection that received all plasmids DNAs except pCDV-L expression vector did not yield detectable amounts of PCR product (see FIG. 4B, lane 1). Furthermore, no PCR product was evident if the reverse transcription step was omitted (see FIG. 4B, lanes 3, 5, 7).

### Example 5

### Expression of Heterologous Genes from Rescued CDV using the Above Rescue Methodology

a) Expression of the Luciferase Gene

To further evaluate CDV as a potential vector, the CDV genomic cDNA was modified to accept a foreign gene. First, nine nucleotide substitutions were introduced in the region between positions 3330 and 3373 (FIG. 5A, 5B). This introduced three restriction enzyme sites (AatII, FseI and MluI) in the intergenic region between the P and M gene (P/M intergenic region). These sites are unique in the genomic cDNA clone pBS-rCDV+(FIG. SB). Virus containing these base substitutions (rCDV+) was rescued demonstrating that these modifications did not have a significant effect on the viability of the virus (data not shown). The FseI and MluI sites were then used to insert the luciferase reporter gene. FIG. 5B shows the nucleotide substitutions made to the original rCDV plasmid vector (pBS-rCDV) to generate plasmid pBS-rCDV+. The luciferase gene was modified and inserted into plasmid prCDV-mcs (FIG. 5B). The luciferase gene was prepared for cloning by first performing PCR to amplify the coding sequence using plasmid pGL2-control (Promega of Madison, Wis.) as template. The PCR primers (See PCR Primer List below, primers 1 and 2) contained terminal restriction enzyme cleavage sites to allow insertion of the amplified reporter gene between the FseI and MluI sites in prCDV-mcs (FIG. 5B). The 5′ PCR primer (primer 1) also contained additional sequences that were equivalent to a synthetic copy of the CDV P/M intergenic transcriptional control sequence. PCR amplification of the luciferase coding sequence with these primers produced a luciferase gene containing the P/M intergenic transcriptional control sequence and an FseI site fused to the 5′ end, and a MluI site at the 3′ end. The amplified sequence was cloned into pBS-rCDV-mcs, and subsequent DNA sequence analysis confirmed that the luciferase gene was accurately cloned to produce pBS-rCDV-P/Luc/M (FIG. 5C).

Virus plaques were detected after using the eDNA containing the luciferase gene in a rescue experiment (see FIG. 4A, rCDV-P/Luc/M). Isolates of recovered virus (rCDV-P/Luc/M) were characterized by sequencing RT/PCR-amplified fragments spanning the junctions between CDV sequences and the luciferase gene, and this revealed that the gene was inserted as expected in the recombinant virus (data not shown).

A luciferase assay was performed with extracts made from cells infected by five different isolates of rCDV-P/Luc/M virus (numbers 1-5). Each well of a six-well plate containing Vero cells was infected with different rCDV strains and cell extracts were prepared at approximately 48 h after infection when 75% or more of the monolayer displayed cell fusion. Extracts were diluted 10⁴ fold and 50 μl was analyzed to produce the results shown in the Luciferase Table below. The negative control samples were analyzed undiluted. These included a mock infection and infections performed with rCDV and rCDV-mcs virus. When the rCDV-P/Luc/M viruses were rescued, a negative control transfection was performed in parallel that lacked L expression plasmid (no pCDV-L). Cell lysate from this parallel mock rescue was used to perform a mock infection that also produced only background levels of luciferase activity. As shown in the Luciferase table below, relatively high levels of luciferase activity were observed in cells infected with different isolates of rCDV-P/Luc/M recovered from independent transfections (see the Table, numbers 1-5). Negative controls yielded very low background levels of luciferase (rCDV, rCDV+, no L plasmid).

Luciferase Table

| | | Luciferase Activity |
|---|---|---|
| Sample | Infection | (relative light units) |
| 1 | Mock | 0 |
| 2 | No pCDV-L | 85 |
| 3 | RCDV-P/Luc/M-1 | 160,909 |
| 4 | RCDV-P/Luc/M-2 | 183,096 |
| 5 | RCDV-P/Luc/M-3 | 170,532 |
| 6 | RCDV-P/Luc/M-4 | 132,221 |
| 7 | RCDV-P/Luc/M-5 | 287,520 |
| 8 | RCDV-mcs | 0 |
| 9 | RCDV | 0 |

b) Expression of the Canine Parvovirus (CPV) VP2 Gene

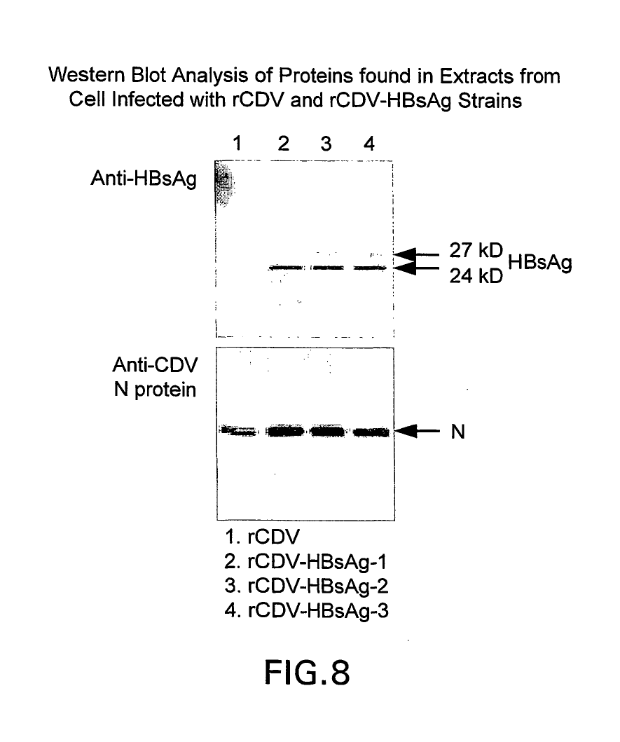

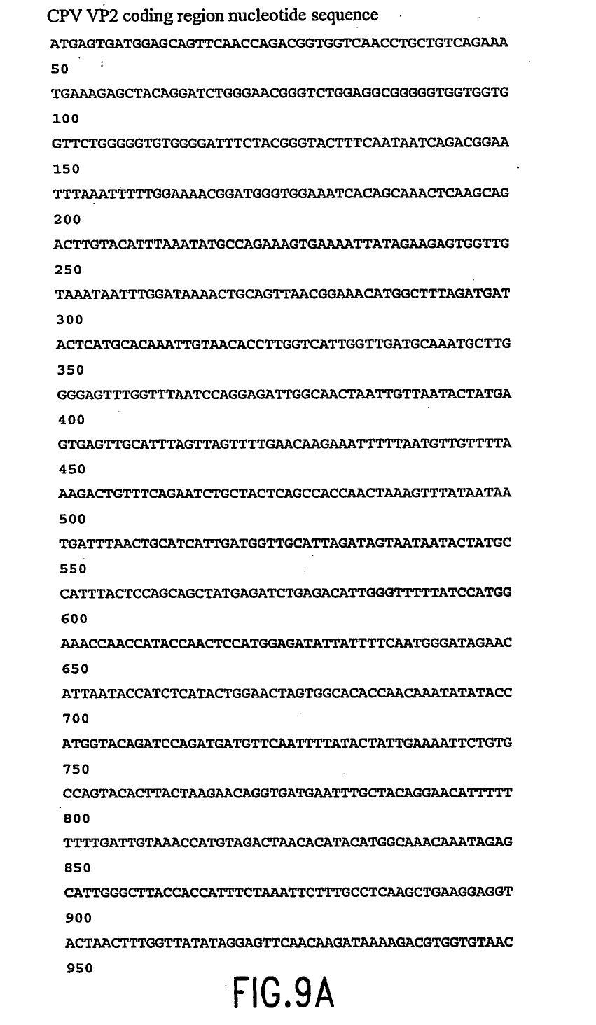

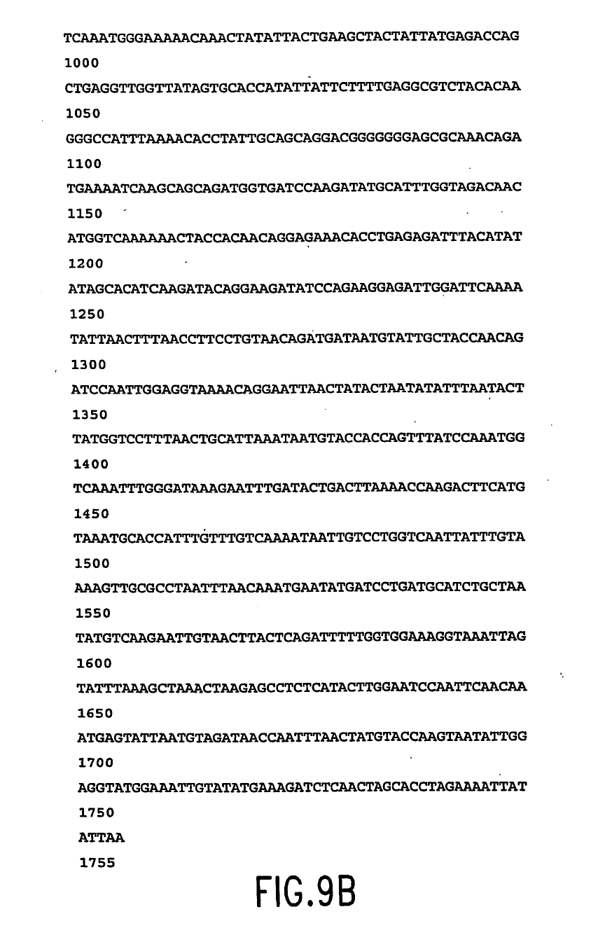

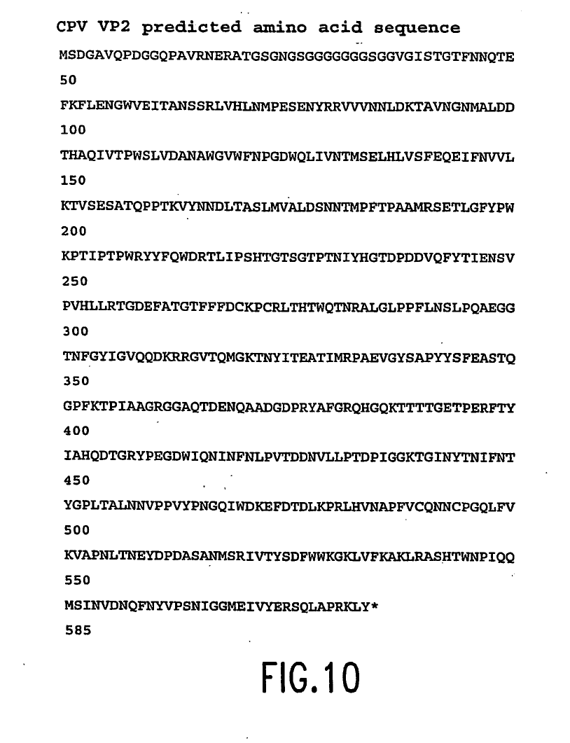

The CDV genornic plasinid containing the CPV VP2 gene (See FIG. 5D and the Flowchart below) was generated. CPV genomic DNA used for cloning the VP2 gene was prepared from a CPV vaccine strain, FD99 (which is the CPV strain isolated from the canine vaccine DURAMUNE® MAX of Fort Dodge Laboratories, Ft. Dodge, Iowa) by proteinase K digestion and organic extraction procedures. The VP2 coding sequence was amplified by PCR using a 5′ primer (primer 4) that contained sequences homologous to the 5′ end of the VP2 coding sequence in addition to sequences equivalent to the CDV P/M intergenic transcriptional control sequence (primer 3). Both the 5′ and the 3′ primers (primers 3 and 4) also contained terminal restriction sites used for insertion of the amplified VP2 coding sequence into plasmid prCDV-mcs as described above. Before cloning the amplified VP2 DNA, a portion of the DNA was used directly for DNA sequence analysis. This provided DNA sequence data for the VP2 gene that was free of any potential nucleotide changes introduced during subsequent cloning steps. Next, the remainder of the VP2 PCR product was used for cloning the gene into a standard cloning vector (pBSK(+)). The nucleotide sequence of the cloned VP2 gene and the attached CDV P/M intergenic transcriptional control sequence was then determined by DNA sequencing using dye-terminator cycle sequencing (cycle sequencing reagents from Applied Biosystems, Foster City, Calif.) and an automated sequencer (Applied Biosystems 377, Foster City, Calif.) (See FIG. 9 for the nucleotide sequence, SEQ ID NO. 39, and FIG. 10 for the amino acid sequence, SEQ ID NO. 40) before it was transferred into the CDV genornic DNA clone (prCDV-mcs) between the P and M genes to generate plasmid pBS-rCDV-VP2 (FIG. 8E). Several viral isolates were rescued from independent transfections using plasmid pBS-rCDV-VP2. Analysis of viral genomic RNA by reverse transcription and PCR amplification (RT/PCR) amplification using primers 7 and 8 (below) revealed that these strains did contain the VP2 gene.

In order to confirm that the rCDV-VP2 viruses express the VP2 protein, one can use a polyclonal antibody, which can be prepared by conventional means. VP2 expression is determined by Western blotting (2) for reactivity to VP2. Briefly, dog kidney cells infected with CPV as a positive control were lysed by boiling in Laenunli buffer (Bio-Rad Laboratories, Hercules, Calif.). Proteins in the crude cell extract were electrophoresed in a 12% polyacrylamide gel then electrophoretically transferred to a nitrocellulose membrane. The membrane was treated with blocking buffer (phospate-buffered saline plus 5% dry milk(Bio-Rad Laboratories, Hercules, Calif.)) then reacted with dog serumn, containing anti-CPV antibodies, diluted in blocking buffer. Antigen-antibody binding was detected using a peroxidase-labeled anti-dog secondary antibody (Sigma, St. Louis, Mo.) and chemiluminesce substrate (SuperSignal West Pico Chemiluminescent Substrate, Pierce, Rockford, Ill.). The Western blot assay revealed that the dog antiserum reacted specifically with a 65 kilodalton protein from cells infected with CPV; the relative mobility of this 65 kD polypeptide was consistent with the expected size of VP2. This methodology can be used to confirm that this same polypeptide species is present in cells infected with rCDV-VP2 strains.

c) Expression of the Hepatitis B Virus Surface Antigen Gene HBsAg

Isolates of rCDV containing the surface antigen gene from Hepatitis B virus (HBV) were rescued using plasmid prCDV-HBsAg (FIG. 8E). Plasmid p BS-rCDV-HBsAg was prepared by inserting the HBsAg coding sequence between the FseI and MluI sites of prCDV-mcs as per examples (a) and (b) above. The HBsAg gene was amplified by PCR from a cloned HBV genome (strain ayw; Genbank accession V01460; (75)) using primers 5 and 6 (see below).

Several independently rescued recombinant strains of CDV containing the HbsAg gene were isolated using plasmid pBS-rCDV-HBsAg. Viral genomic RNA from the rCDV-HBsAg isolates (rCDV-HBsAg-1, -2 and -3) was analyzed by RT-PCR using gene-specific primers (Primers 7 and 8) to confirm that the recombinant isolates contained the HBsAg gene. After confirming that the recombinant viruses contained the FBsAg gene, Western blot analysis was performed to ensure that the HBsAg gene was expressed. As shown in FIG. 8, Western blot analysis revealed that a 24 and 27 kD. The 27 kD form of the HbsAg strain is a glycosylated form of the protein (76). Cell extracts infected with recombinant CDV lacking the BBV gene did not react with the antibody (Fitzgerald Industries International Inc., Concord, Mass.). As a control, the blot was stripped and probed with anti-CDV N protein antibody (VMRD, Inc, Pullman, Wash.), confirming that all extracts were prepared from cells infected with CDV.

PCR Primer List

(SEQ ID NO. 31)

```
                1. Luciferase gene 5′ end
        5′-TACTGGCCGGCCATTATAAAAAACTTAGGACACAAGAGCC
TAAGTCCGCTGCCACCATGGAAGACGCCAAAAACAT-3′
```

The CDV gene-end/gene-start signal is underlined and the FseI site is italicized. A Kozak (77) translational control consensus sequence was added (GCCACC) preceding the luciferase ATG initiator codon (bold).

2. Luciferase 3′ end

5′-TTTTACGCGITTACAATTTGGACTTTCCGC-3′ (SEQ ID NO 32 ) MluI site is italicized.

(SEQ ID NO. 33)

```
        3. CPV VP2 5′ end
        TACTGGCCGGCCATTATAAAAAACTTAGGACACAAGAGCCTAA
GTCCGCTGCCACCATGAGTGATGGAGCAGTTCAAC
```

See description of primer 1.

| | |
|---|---|
| 4. CPV VP2 3′ end | |
| TTTTACGCGTTTAATATAATTTTCTAGGTGC | (SEQ ID NO. 34) |

MluI site is italicized.

(SEQ ID NO 35)

```
        5. HBsAg 5′ end
        TACTGGCCGGCCATTATAAAAAACTTAGGACACAAGAGCCTAA
GTCCGCTGCCACCATGGAGAACATCACATCAGGAT
```

See description of primer 1.

(SEQ ID NO. 36)

```
        6. HBsAg 3′ end
        TTTTACGCGTTTATCAGCTGGCATAGTCAGGCACGTCATAAGGA
TAGCTAATGTATACCCAAAGACA
```

MluI site is italicized.

7. 5′ of FseI site

| | |
|---|---|
| ATAACATGCTGGCTCTGCTC | (SEQ ID NO. 37) |

5′ Primer used for PCR analysis of genes inserted into genome of recombinant CDV strains. Specific for CDV sequences flanking the 5′ end of the foreign gene.

8. 3′ of MluI site

GCTAGTCAGGAGAACCATGT (SEQ ID NO. 38)

3′ Primer used for PCR analysis of genes inserted into the genome of recombinant CDV strains. Specific for CDV sequences flanking the 3′ end of the foreign gene.

### Flow Chart for the Development of a CDV Expression Vector that Contains the CPV VP2 Gene

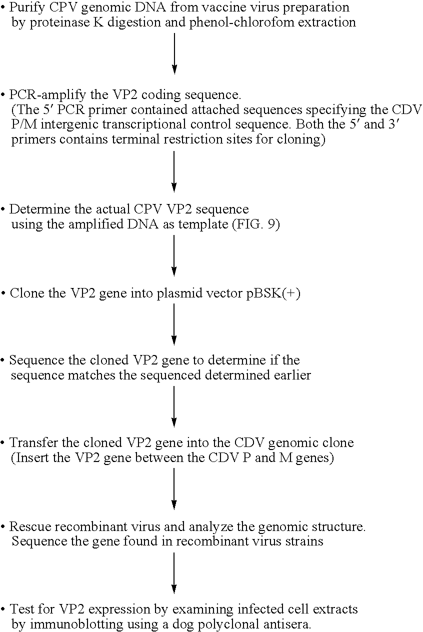

Provided below are a list of references which are incorporated herein.

### References

1. Appel, M. J. G. 1999. Forty years of canine vaccination. Adv. Vet. Med. 41:309-324.

2. Ausubel, F. M., R. Brent, R. E. Kingston, D. D. Moore, J. G. Siedman, J. A. Smith, and K. Struhl (ed.). 1987. Current Protocols in Molecular Biology. Greene Publishing Associates and Wiley Interscience, New York.

3. Ball, L. A., C. R. Pringle, B. Flanagan, V. P. Perepelitsa, and G. W. Wertz. 1999. Phenotypic consequences of rearranging the p, M and G genes of vesicular stomatitis virus. J. Virol. 73:4705-4712.

4. Barrett, T. 1999. Morbillivirus infection, with special emphasis on morbilliviruses of carnovores. Vet. Micro. 69:3-13.

5. Been, M. D., and G. S. Wickham. 1997. Self-cleaving ribozymes of hepatitis delta virus RNA. Eur. J. Biochem. 247:741-753.

6. Calain, P., and L. Roux. 1993. The rule of six, a basic feature for efficient replication of Sendai virus defective interfering RNA. J. Virol. 67:4822-4830.

7. Carmichael, L. E. 1999. Canine viral vaccines at a turning point—a personal perspective. Adv. Vet. Med 41:289-304.

8. Chen, C., and H. Okayama. 1987. High-efficiency transformation of mammalian cells by plasmid DNA. Mol. Cell Biol. 7:2745-2752.

9. Chomczynski, P., and N. Sacchi. 1987. Single-step method of RNA isolation by acid guanidinium thiocyanate-phenol-chloroform extraction. Analytical Biochem. 162:156-159.

10. Conzelmann, K. K. 1998. Nonsegmented negative-strand RNA viruses: genetics and manipulation of viral genomes. Annual Review of Genetics 32:123-162.

11. de Swart, R. L., T. C. Harder, P. S. Ross, H. W. Vos, and A. D. M. E. Osterhaus. 1995. Morbilliviruses and morbillivirus disease of marine mammals. Infectious Agents Disease 4:125-130.

12. Durbin, A. P., J. M. McAuliffe, P. L. Collins, and B. R. Murphy. 1999. Mutations in the C, D, and V open reading frames of human parainfluenza virus type 3 attenuate replication in rodents and primates. Virology 261:319-330.

13. Escoffier, C., S. Mainie, S. Vincent, Muller, C. P., M. A. Billeter, and D. Gerlier. 1999. Nonstructural C protein is required for efficient measles virus replication in human peripheral blood cells. J. Virol. 73:1695-1698.

14. Fraser, D. W. 1997. Paget's disease of bone. Curr. Opinion Rheumatology 9:347-354.

15. Garcin, D., M. Itoh, and D. Kolakofsky. 1997. A point mutation in the Sendai virus accessory C proteins attenuates virulence for mice but not virus growth in cell culture. Virology 238:424-431.

16. Griffin, H. G., and A. M. Griffin. 1993. DNA sequencing. Recent innovations and future trends. Appl. Biochem. Biotech. 38:147-159.

17. Haig, D. A. 1956. Canine distemper-immunization with avianised virus. Onderstepoort J. Vet. Res. 27:19-53.

18. Harder, T. C., and A. D. M. E. Osterhaus. 1997. Canine distemper virus—a morbillivirus in search of a new host. Trends Micro. 5:120-124.

19. Hodge, M. J., and C. Wolfson. 1997. Canine distemper virus and multiple sclerosis. Neurology 49 (Suppl.):S62-S69.

20. Hruby, D. E., and E. M. U. Wilson. 1992. Use of fluorescent chloramphenicol derivative as a substrate for chloramphenicol acetyltransferase assays. Methods Enzymol. 216:369-376.

21. Juhasz, K., B. R. Murphy, and P. L. Collins. 1999. The major attenuating mutations of the respiratory syncytial virus vaccine candidate cpts530/1009 specify temperature-sensitive defects in transcription and replication and a non-temperature-sensitive alteration in mRNA termination. J. Virol. 73:5176-5180.

22. Kato, A., K. Kiyotani, Y. Sakai, T. Yoshida, and Y. Nagai. 1997. The paramyxovirus, Sendai virus, V protein encodes a luxury function required for viral pathogenesis. EMBO J. 16:578-587.

23. Kolakofsky, D., T. Pelet, D. Garcin, S. Hausmann, J. Curran, and L. Roux. 1998. Paramyxovirus RNA synthesis and the requirement for hexamer genome length: the rule of six revisited. J. Virol. 72:891-899.

24. Kretz, K., W. Callen, and V. Hedden. 1994. Cycle sequencing. Genome Res. 3:S101-S112.

25. Lamb, R. A., and D. Kolakofsky. 1996. Paramyxoviridae: the viruses and their replication. In B. N. Fields, D. M. Knipe, P. M. Howley, R. M. Chanock, T. O. Monath, J. L. Melnick, B. Roizman, and S. E. Straus (ed.), Fields Virology, Third ed. Lippncott-Raven Publishers, Philadelphia.

26. Maniatis, T., E. F. Fritsch, and J. Sambrook. 1982. Molecular Cloning: a laboratory manual. Cold Spring Harbor Laboratory, Cold Spring Harbor, N.Y.

27. Mcllhatton, M. A., M. D. Curran, and B. K. Rima. 1997. Nucleotide sequence analysis of the large (L) genes of phocine distemper virus and canine distemper virus (corrected). J. Gen. Virol. 78:571-576.

28. Mee, A. P., and P. T. Sharpe. 1993. Dogs, distemper and Paget's disease. BioEsaays 15:783-789.

29. Moss, B., O. Elroy-Stein, T. Mizukami, W. A. Alexander, and T. R. Fuerst. 1990. New mammalian expression vectors. Nature 348:91-92.

30. Murphy, F. A. 1996. Virus Taxonomy. In B. N. Fields, D. M. Knipe, P. M. Howley, R. M. Chanock, T. O. Monath, J. L. Melnick, B. Roizman, and S. E. Straus (ed.), Fields Virology, Third ed. Lippncott-Raven Publishers, Philadelphia.

31. Nagai, Y. 1999. Paramyxovirus replication and pathogenesis. Reverse genetics transforms understanding. Rev. Med. Virol. 9:83-99.

32. Nagai, Y., and A. Kato. 1999. Paramyxovirus reverse genetics is coming of age. Microbiol. Immunol. 43:613-624.

33. Oglesbee, M. J., H. Kenney, T. Kenney, and S. Krakowka. 1993. Enhanced production of Morbillivirus gene-specific RNAs following induction of the cellular stress response in stable persistent infection. Virology 192:556-567.

34. Pardo, M. C., J. E. Bauman, and M. Mackowiak. 1997. Protection of dogs against canine distemper by vaccination with a canarypox virus recombinant expressing canine distemper virus fusion and hemagglutinin glycoproteins. American J. Vet. Res. 58:833-836.

35. Parks, C. L., R. A. Lerch, P. Walpita, M. S. Sidhu, and S. A. Udem. 1999. Enhanced measles virus cDNA rescue and gene expression after heat shock. J. Virol. 73:3560-3566.

36. Parks, C. L., R. A. Lerch, P. Walpita, H. Wang, M. S. Sidhu, and S. A. Udem. 2000. Comparison of Predicted Amino Acid Sequences from Measles Virus Strains in the Edmonston Vaccine Lineage. J. Virol Submitted.

37. Patterson, J. B., D. Thomas, H. Lewicki, M. A. Billeter, and M. B. A. Oldstone. 2000. V and C proteins of measles virus function as virulence factors in vivo. Virology 267:80-89.

38. Pattnaik, A. K., L. A. Ball, A. W. LeGrone, and G. W. Wertz. 1992. Use of T7 phage polymerase and hepatits delta virus ribozyme sequences for cDNA synthesis and processing. Cell 69:1011-20.

39. Pekosz, A., and R. A. Lamb. 1999. Reverse genetics of negative-strand RNA viruses: Closing the circle. Proc. Natl. Acad. Sci. USA 96:8804-8806.

40. Radecke, F., and M. A. Billeter. 1997. Reverse genetics meets the nonsegmented negative-strand RNA viruses. Rev. Med. Virology 7:49-63.

41. Radecke, F., P. Spielhofer, H. Schmeider, K. Kaelin, M. Huber, C. Dotsch, G. Christiansen, and M. A. Billeter. 1995. Rescue of measles viruses from cloned DNA. EMBO J. 14:5773-5784.

42. Roberts, A., and J. K. Rose. 1998. Recovery of negative-strand RNA viruses from plasmid DNAs: a positive approach revitalizes a negative field. Virology 247:1-6.

43. Schneider, H., P. Spielhofer, K. Kaelin, C. Dotsch, F. Radeke, G. Sutter, and M. A. Billeter. 1997. Rescue of measles virus using a replication-deficient vaccinia-T7 vector. J. Virological Methods 64:57-64.

44. Schnell, M. J., T. Mebatsion, and K.-K. Conzelmann. 1994. Infectious rabies viruses from cloned eDNA. EMBO J. 13:4195-4203.

45. Sidhu, M. S., J. Chan, K. Kaelin, P. Spielhofer, F. Radecke, H. Schnieder, M. Masurekar, P. C. Dowling, M. A. Billeter, and S. A. Udem. 1995. Rescue of synthetic mealses virus minireplicons: measles genomic termini direct efficient expression and propagation of a reporter gene. Virology 208:800-807.

46. Sidhu, M. S., W. Husar, S. D. Cook, P. C. Dowling, and S. A. Udem. 1993. Canine distemper terminal and intergenic non-protein coding nucleotide sequences: completion of the entire CDV genome sequence. Virology 193:66-72.

47. Sidhu, M. S., J. P. Menonna, S. D. Cook, P. C. Dowling, and S. A. Udem. 1993. Canine distemper virus L gene: Sequence and comparison with related viruses. Virology 193:50-65.

48. Sixt, N., A. Cardoso, A. Vallier, J. Fayolle, R. Buckland, and T. F. Wild. 1998. Canine distemper virus DNA vaccination induces humoral and cellular immunity and protects againast a lethal intracerebral challenge. J. Virology 72:8472-8476.

49. Skiadopoulos, M. H., A. P. Durbin, J. M. Tatem, S.-L. Wu, M. Paschalis, T. Tao, P. L. Collins, and B. R. Murphy. 1998. Three amino acid substitutions in the L protein of the human parifluenza virus type 3 cp45 live attenuated vaccine candidate contribute to its temperature-sensitive and attenuation phenotype. J. Virol. 72:1762-1768.

50. Skiadopoulos, M. H., S. Surman, J. M. Tatem, M. Paschalis, S.-L. Wu, S. A. Udem, A. P. Durbin, P. L. Collins, and B. R. Murphy. 1999. Identification of mutations contributing to the temperature-sensitive, cold-adapted, and attenuated phenotypes of live-attenuated cold-passage 45 (cp45) human parainfluenza virus 3 candidate vaccine. J. Virol. 73:1374-1381.

51. Stephenson, C. B., J. Welter, S. R. Thaker, J. Taylor, J. Tartaglia, and E. Paoletti. 1997. Canine distemper virus (CDV) infection of ferrets as a model for testing morbillivirus vaccine strategies: NYVAC- and ALVAC-based CDV recombinants protect against symptomatic infection. J. Virology 71:1506-1513.

52. Summers, B. A., and J. G. Appel. 1994. Aspects of canine distemper virus and measles virus encephalomyelitis. Neuropath. Applied Neurobiol. 20:525-534.

53. Tober, C., M. Seufert, H. Schneider, M. A. Billeter, I. C. D. Johnston, S. Niewiesk, Ter Meulen., and S. Schneider-Schaulies. 1998. Expression of measles virus V protein is associated with pathogenicity and control of viral RNA synthesis. J. Virol. 72:8124-8132.

54. Tognon, M., E. M. Cattozzo, S. Bianchi, and M. G. Romanelli. 1996. Enhancement of HSV-DNA infectivity, in Vero and RS cells, by a modified calcium-phosphate transfection technique. Virus Genes 12:193-197.

55. Udem, S. A. 1984. Measles virus: conditions for the propagation and purification of infectious virus in high yield. J. Virological Methods 8:123-136.

56. Valsamakis, A., H. Schneider, P. G. Auwaerter, H. Kaneshima, M. A. Billeter, and D. E. Griffin. 1998. Recombinant measles viruses with mutations in the C, V, or F gene have altered growth phenotypes in vivo. J. Virol. 72:7754-7761.

57. Vandevelde, M., and A. Zubriggen. 1995. The neurobiology of canine distemper virus infection. Vet. Micro.:271-280.

58. Wertz, G. W., V. P. Perepelitsa, and L. A. Ball. 1998. Gene rearrangement attenuates expression and lethality of a nonsegmented negative strand RNA virus. Proc. Natl. Acad. Sci. USA 95:3501-3506.

59. Whitehead, S. S., C.-Y Firestone, P. L. Collins, and B. R. Murphy. 1998. A single nucleotide substitution in the transcription start signal of the M2 gene of respiratory syncitial virus vaccine candidate cpts248/404 is the major determinant of temperature-sensitive and attenuation phenotypes. Virology 247:232-239.

60. Whitehead, S. S., C. Y. Firestone, R. A. Karron, J. E. Crowe, W. R. Elkins, P. L. Collins, and B. R. Murphy. 1999. Addition of a missense mutation present in the L gene of respiratory syncytial virus (RSV) cpts530/1030 to RSV vaccine candidate cpts248/404 increases its attenuation and temperature sensitivity. J. Virology 73:871-877.

61. Wyatt, L. S., B. Moss, and R. S. 1995. Replication-deficient vaccinia virus encoding bacteriophage T7 RNA polymerase for transient gene expression in mammalian cells. Virology 210:202-205.

62. Young, S. L., L. Barbera, A. H. Kaynard, R. P. Haugland, H. C. Kang, M. Brinkley, and M. H. Melner 1991. A non-radioactive assay for transfected chloramphenicol acetyltransferase activity using fluorescent substrates. Analytical Biochem. 197:401-407.

63. Baron, M. D., and Barrett, T. (1997). Rescue of rinderpest virus from cloned cDNA. J Virol, 71, 1265-1271.

64. Clarke, David K., Sidhu, Mohinderijit S., Johnson, Erik J., and Udem, Stephen A. (2000). Rescue of mumps Virus from cDNA. Journal of Virology. 74: 4831-483S.

65. Collins, P. L., Hill, M. G., Camargo, E., Grosfield, H., Chanock, R. M., and Murphy, B. R.(1995). Production of infectious human respiratory syncytial virus from cloned cDNA confirns an essential role for the transcription elongation factor from the 5″ proximal open reading frame of the M2 mRNA in gene expression and provides a capability for vaccine development. Proc. Natl. Acad. Sci. USA 92; 11563-11567.

66. Garcin, D., Pelet, T., Calain, P., Sakai, Y., Shioda, T., Roux, L., Curran, J., Kolakofsky, D. (1995). A highly recombinogenic system for the recovery of infectious Sendia paramyxovirus from cDNA:; generation of a novel copyback nondefective interfering virus. EMBO J. 14: 6087-6094.

67. He, B., Paterson, R. G., Ward, C. D. and Lamb, R. A. (1997). Recovery of infectious SV5 from cloned DNA and expression of a foreign gene. Virology, 237: 249-260.

68. Hoffinan, M. A., and Banerjee, A. K. (1997). An infectious clone of human parainfluenza virus type 3. J Virol. 71; 4272-4277.

69. Kyte, J and Doolittle, R. F. (1992). J. Mol. Biol. 157: 105-137.

70. Lawson, N., Stillman, E., Whitt, M., and Rose, J. (1995). Recombinant vesicular stomatitis viruses from DNA. Proc. Natl. Acad. Sci. USA 92; 4471-4481.

71. Sambrook, Fritsch and Maniatis. Molecular Cloning—A Laboratory Manual. Cold Spring Press (Cold Spring Harbor, N.Y.). 1989.

72. Sutter, G., Ohlmann, M. and Erfle, V. Non-replicating vaccinia vector efficiently expressing bacteriophage T7 RNA polymerase.(1995-August). FEBS Letters 371 (1): 9-12.

73. Whelan, S. P., Ball, L. A., Barr, J. N. and Wertz, G. T. (1995). Efficient recovery of infectious vesicular stomatitis virus entirely from cDNA clones. Proc. Natl. Acad. Sci. USA 92; 8388-8392.

74. Wyatt, L.S., Moss, B. and Rozenblat, S. (1995-June). Replication-deficient vaccinia virus encoding bacteriophage T7 RNA polymerase for transient gene expression in mammalian cells. Virology. 210 (1); 202-5.

75. Galibert, F., E. Mandart, F. Fitoussi, P. Tiollais, and P. Charnay. (1979). Nucleotide sequence of the hepatitis B virus genome (subtype ayw) cloned in E. coli. Nature. 281:646-650.

76. Ganem, D. (1996). Hepadnaviridae and their replication, p. 2703-2737. ln B. N. Fields and D. M. Knipe and P. M. Howley and R. M. Chanock and T. O. Monath and J. L. Melnick and B. Roizmnan and S. E. Straus (ed.), Fields Virology, Third ed. Lippncott-Raven Publishers, Philadelphia.

77. Kozak, M. (1989). The scanning model of translation: an update. J. Cell Biol. 108:229-241.

1. A method for producing a recombinant canine distemper virus comprising;
in at least one host cell, conducting transfection or transformation, in media, of a rescue composition which comprises (i) a transcription vector comprising an isolated nucleic acid molecule which comprises a polynucleotide sequence encoding a genome or antigenome of canine distemper virus, or variant polynucleotide sequence thereof, and (ii) at least one expression vector which comprises one more isolated nucleic acid molecule(s) comprising a polynucleotide sequence encoding the trans-acting proteins (N, P and L) necessary for encapsidation, transcription and replication; under conditions sufficient to permit the co-expression of said vectors and the production of the recombinant virus.

2. The method of claim 1 further comprising harvesting the recombinant virus.

3. The method of claim 1 wherein the isolated nucleic acid molecule encoding a genome or antigenome of canine distemper virus is a chimera of more than one genome or anti-genome source.

4. The method of claim 1 wherein the isolated nucleic acid molecule encoding a genome or antigenome of canine distemper virus comprises the polynucleotide sequence of SEQ. ID NOS. 1, 2 or 3.

5. The method of claim 1 wherein the isolated nucleic acid molecule, encoding a genome or antigenome of canine distemper virus, encodes an attenuated virus or an infectious form of the virus.

6. The method of claim 1 wherein the isolated nucleic acid molecule, encoding a genome or antigenome of canine distemper virus, encodes an infectious form of the virus.

7. The method of claim 1 wherein the isolated nucleic acid molecule, encoding a genome or antigenome of canine distemper virus, encodes an attenuated virus.

8. The method of claim 1 wherein the isolated nucleic acid molecule, encoding a genome or antigenome of canine distemper virus, encodes an infectious, attenuated virus.

9. The method of claim 1 wherein the host cell is a eukaryotic cell.

10. The method of claim 1 wherein the host cell is a vertebrate cell.

11. The method of claim 1 wherein the host cell is an avian cell.

12. The method of claim 1 wherein the host cell is derived from a human cell.

13. The method of claim 9 wherein the host cell is derived from a human embryonic cell.

14. The method of claim 12 wherein the host cell is derived from a human embryonic kidney cell, human lung carcinoma and human cervical carcinoma, or animal kidney cells.

15. A recombinant canine distemper virus prepared from the method of claim 1.

16. A composition comprising (i) a recombinant canine distemper virus prepared from the method of claim 1 and (ii) a pharmaceutically acceptable carrier.

17. The method of claim 1 wherein transcription vector farther comprises a 17 RNA polymerase gene.

18. An immunogenic composition comprising an isolated, recombinantly-produced canine distemper virus and a physiologically acceptable carrier.

19. A method for immunizing an animal or human to induce protection against canine distemper virus which comprises administering to the animal or human the immunogenic composition of claim 18.

20. A nucleic acid molecule comprising a polynucleotide sequence encoding a genome or antigenome of canine distemper virus.

21. The nucleic acid molecule of claim 20 comprising a canine distemper virus sequence in positive strand, antigenomic message sense of SEQ ID NO 1.

22. A nucleic acid molecule comprising a polynucleotide sequence encoding one or more proteins of the canine distemper virus.

23. The nucleic acid molecule of claims 20, 21 or 22 wherein said polynucleotide sequence further comprises one or more heterologous nucleotide sequences or one or more heterologous genes.

24. A plasmid comprising a polynucleotide sequence encoding a genome or antigenome of canine distemper virus.

25. A plasmid comprising a polynucleotide sequence encoding one or more proteins of the canine distemper virus.

26. The plasmid of claim 24 wherein the polynucleotide sequence further comprises one or more heterologous nucleotide sequences or one or more heterologous genes.

27. The plasmid of claim 25 wherein said polynucleotide sequence further comprises one or more heterologous nucleotide sequences or one or more heterologous genes.

28. A host cell transformed with at least one plasmid of claims 24-27.

29. A composition comprising an isolated, recombinantly-produced canine distemper virus and a physiologically acceptable carrier; wherein the canine distemper virus expresses at least one heterologous polynucleotide.

30. The immunogenic composition of claim 18 wherein the canine distemper virus expresses at least one heterologous polynucleotide encoding an antigen.

31. The immunogenic composition of claim 18 further comprising at least one antigen to a pathogen other than canine distemper virus.

32. The immunogenic composition of claim 31 wherein at least one antigen is an attenuated RNA virus.

33. The immunogenic composition of claim 18 further comprising at least one antigen to pathogen which infects canines.

34. The immunogenic composition of claim 31 wherein at least one antigen is an antigen to one or more viruses selected from the group consisting of rabies virus, canine parvovirus, canine parvovirus 2, canine corona virus, canine adenovirus type 1, canine adenovirus type 2, and canine parainfluenza virus.

35. The immunogenic composition of claim 18 further comprising at least one antigen to pathogen which infects humans.

36. The immunogenic composition of claim 31 wherein at least one antigen, of a pathogen other than canine virus, is expressed from the recombinantly produced canine distemper virus.

37. The immunogenic composition of claim 31 wherein at least one antigen is an antigen to one or more canine paroviruses.

38. A nucleotide sequence comprising the sequence of a cDNA clone of a recombinant canine distemper virus.

39. The plasmid of claim 26 wherein the heterologous nucleotide sequence is inserted within the canine distemper virus genome sequence as a single transcriptional unit.

40. The plasmid of claim 26 wherein the heterologous nucleotide sequence is inserted within the canine distemper virus genome sequence as one or more monocistronic transcriptional units.

41. The plasmid of claim 26 wherein the heterologous nucleotide sequence is inserted within the canine distemper virus genome sequence as at least one polycistronic transcriptional unit, which may contain one or more ribosomal entry sites.

42. A composition comprising an isolated, recombinantly-produced, canine distemper virus produced by a host cell of claim 28, and a physiologically acceptable carrier.

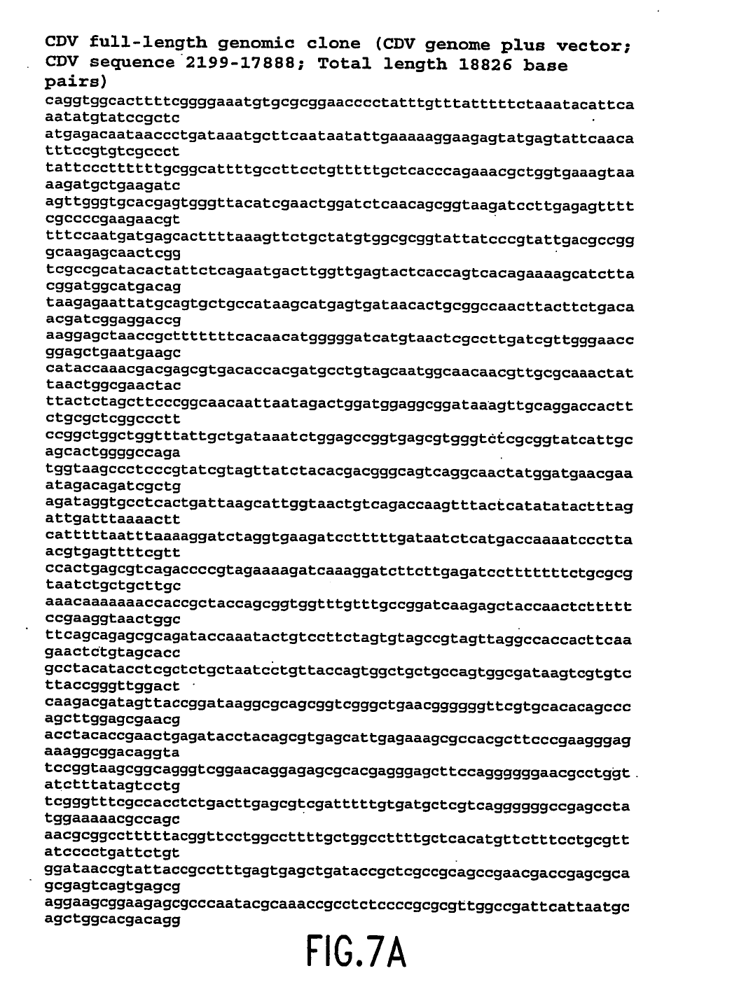

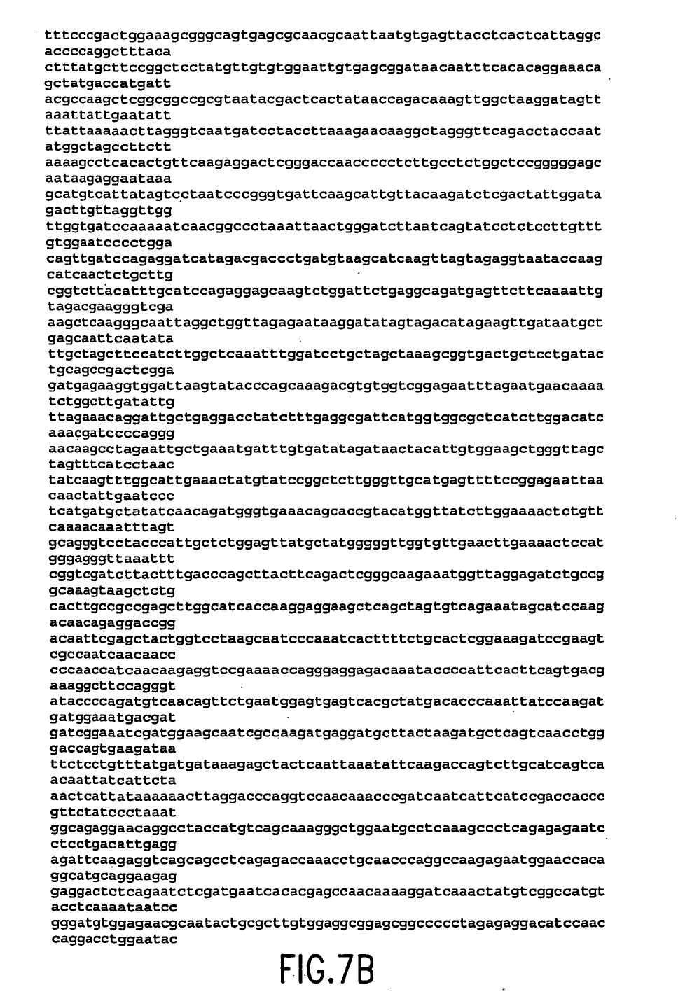

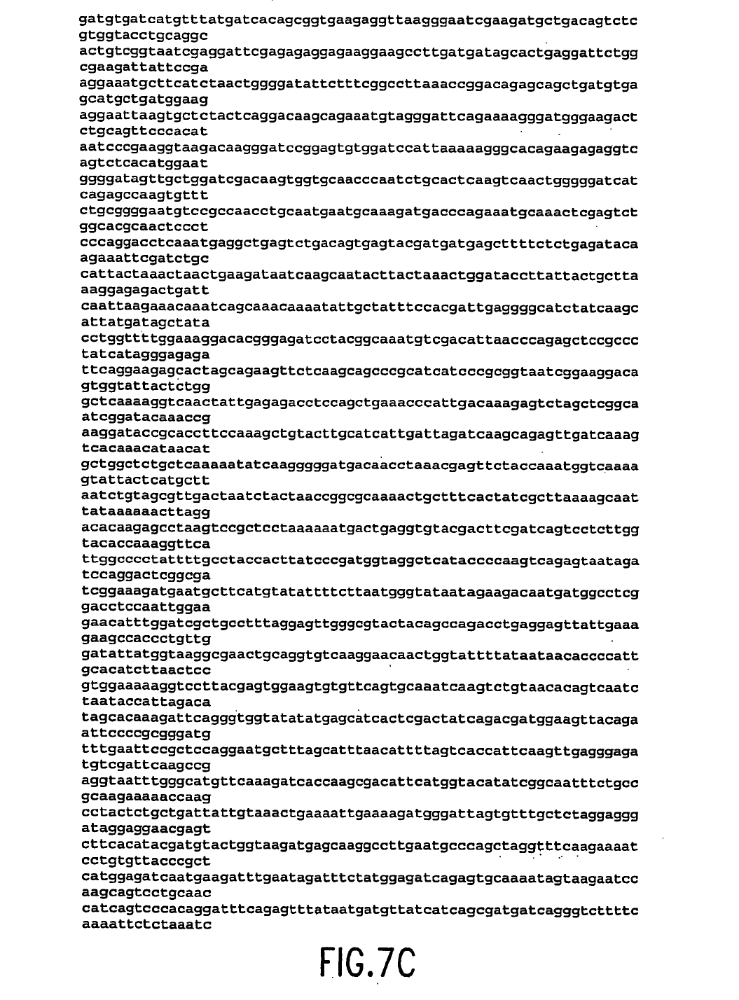

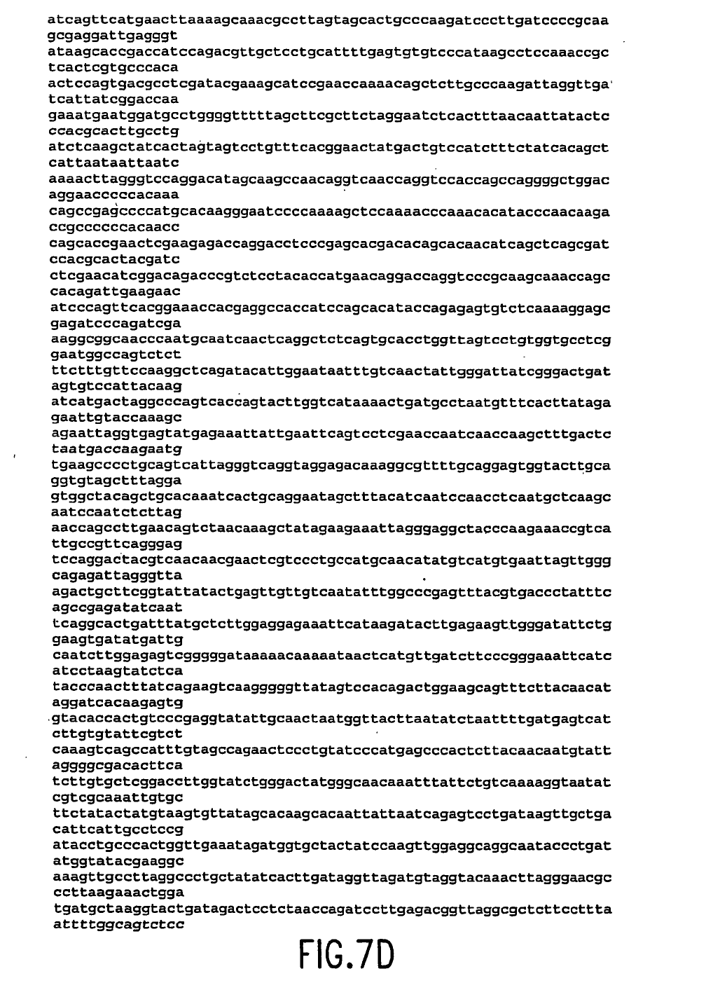

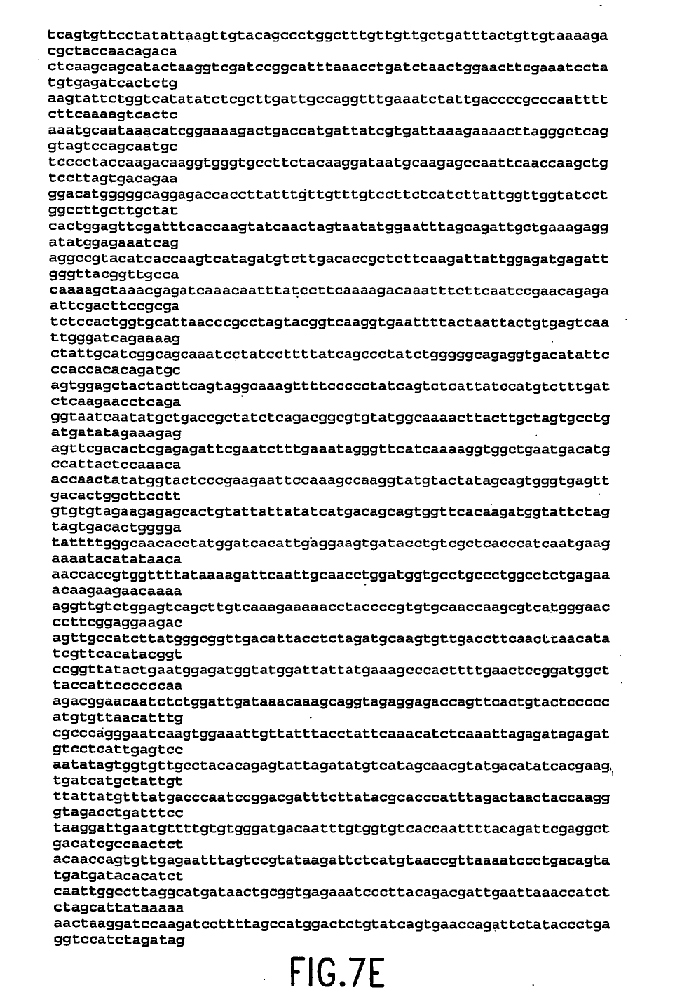

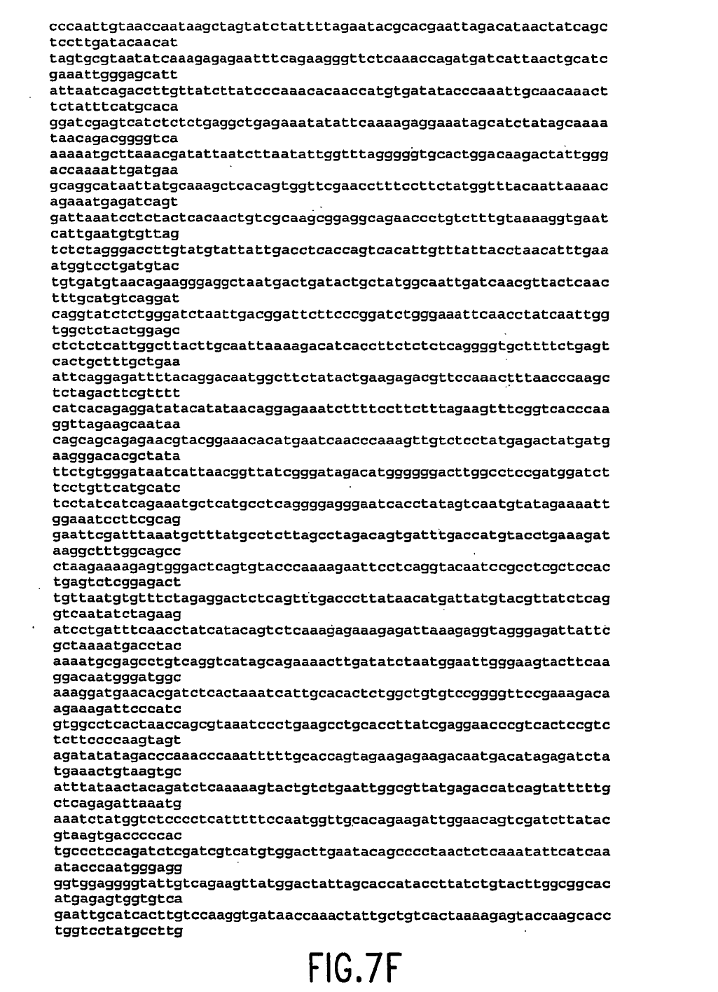

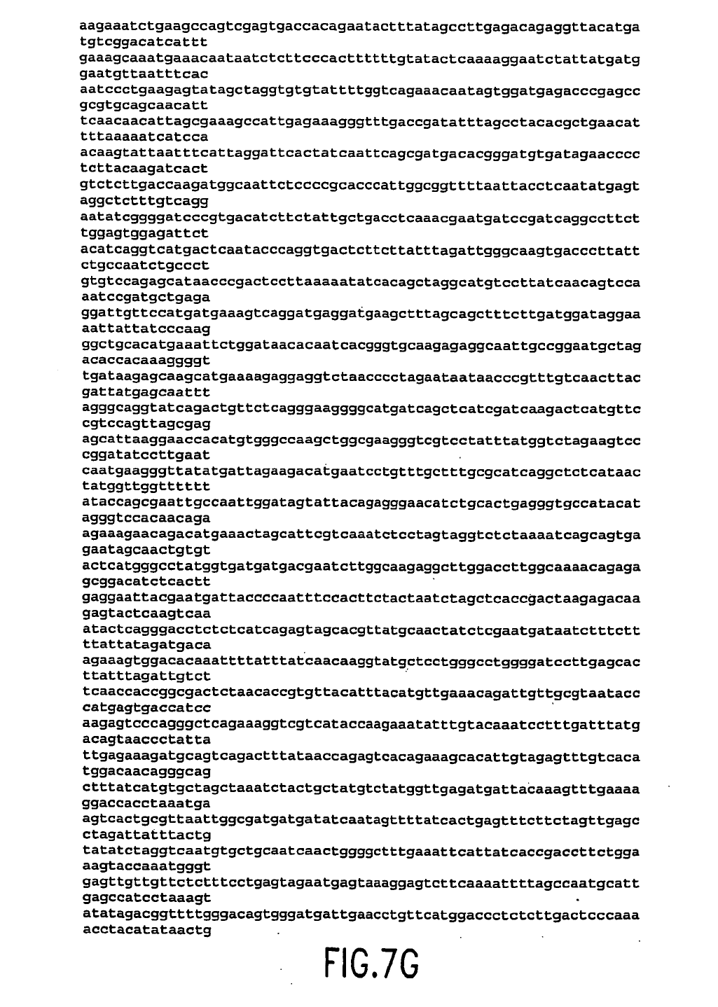

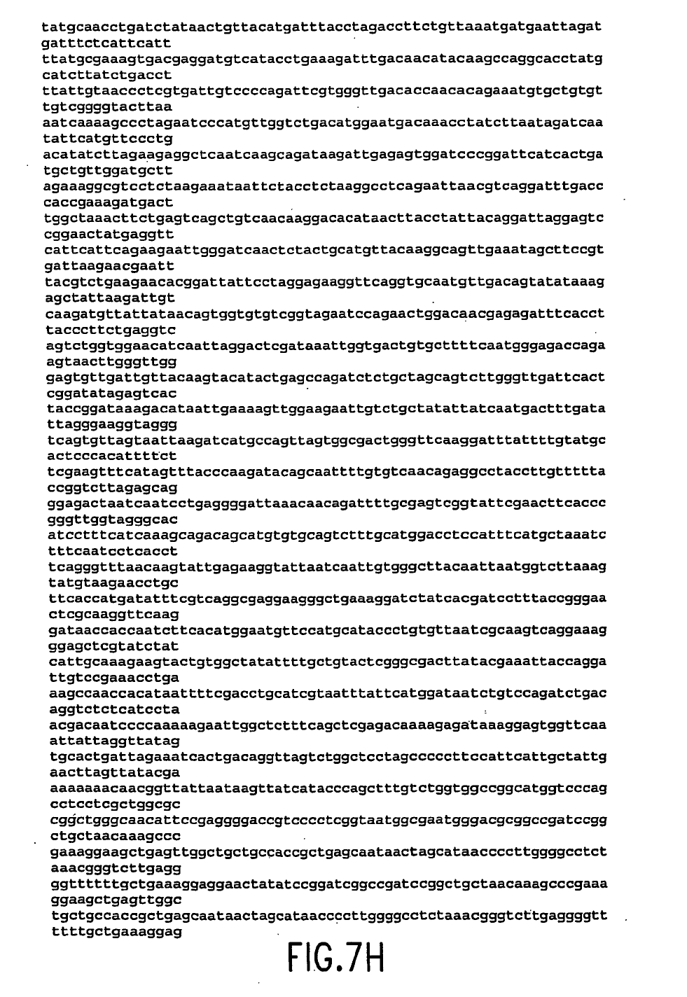

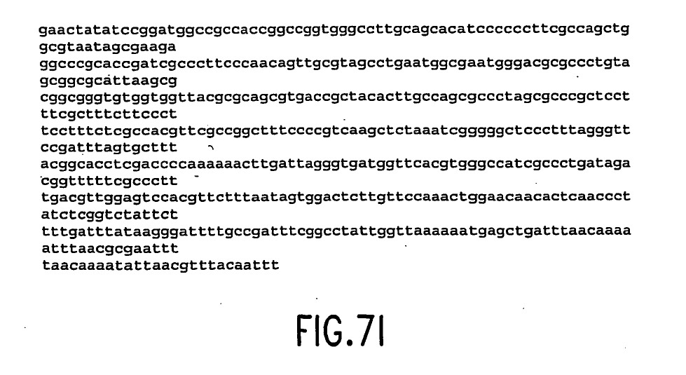

43. A nucleotide sequence comprising the polynucleotide sequence of a cDNA clone of a recombinant canine distemper virus of FIG. 6 (SEQ ID NO. 2) or FIG. 7 (SEQ ID NO. 3).

44. The method of claim 19 wherein the animal is selected from the group consisting of canine, feline, bovine, swine and equine.

45. A method for immunizing an animal or human to induce protection against canine distemper virus which comprises administering to the animal or human an immunogenic composition of claims 18, 30-37.

46. The method of claim 1 wherein the polynucleotide sequence encoding a genome or antigenome of canine distemper virus, or variant polynucleotide sequence thereof, contains at least one mutation of a wild type nucleotide of a canine distemper virus so that such mutation corresponds to a known attenuating mutation in a coding or non-coding region of another non-segmented, negative-sense, single stranded RNA Viruses of the Order Mononegavirales.

47. The method of claim 1 wherein the polynucleotide sequence encoding a genome or antigenome of canine distemper virus, or variant polynucleotide sequence thereof, contains at least one mutation that renders the recombinantly-produced virus replication defective.

48. The method of claim 46 wherein the RNA virus is selected from PIV, RSV, Mumps and Measles.
